(19) United States
(12) Patent Application Publication
(10) Pub. No.: US 2017/0112471 A1
(43) Pub. Date: Apr. 27, 2017

(54) ULTRASOUND DIAGNOSTIC APPARATUS AND ULTRASOUND SIGNAL PROCESSING METHOD

(71) Applicant: Konica Minolta, Inc., Tokyo (JP)

(72) Inventor: BUMPEI TOJI, Hashima-shi (JP)

(21) Appl. No.: 15/298,714

(22) Filed: Oct. 20, 2016

(30) Foreign Application Priority Data
Oct. 27, 2015 (JP) 2015-210514

(51) Int. Cl. A61B 8/08; A61B 8/00

(52) U.S. Cl. CPC A61B 8/485; A61B 8/5207; A61B 8/5223; A61B 8/4254; A61B 8/54

(57) ABSTRACT

An ultrasound diagnostic apparatus that transmits a push pulse for focusing ultrasonic waves on a specific part of a subject to physically press tissues using an ultrasound probe, repeatedly transmits and receives the ultrasonic waves to and from the subject, and detects a propagation state of shear waves in a region of interest, includes: a push pulse transmission unit that transmits the push pulse; a displacement detection unit that transmits detection waves to the subject, receives reflected detection waves, generates a plurality of received signals in time series, and detects displacement of the tissues; an elasticity measurement unit that analyzes the propagation state of the shear waves and measures elasticity of each tissue; a probe movement detection unit that detects a moving speed of the ultrasound probe; a sequence holding unit that holds a plurality of operation sequences; and a sequence selection unit that selects one operation sequence.

### CROSS REFERENCE TO RELATED APPLICATIONS

The entire disclosure of Japanese Patent Application No. 2015-210514 filed on Oct. 27, 2015 including description, claims, drawings, and abstract are incorporated herein by reference in its entirety.

### BACKGROUND OF THE INVENTION

Field of the Invention

The present invention relates to an ultrasound diagnostic apparatus and an ultrasound signal processing method, and more particularly, to the measurement of the hardness of tissues using shear waves.

Description of the Related Art

In recent years, an ultrasound diagnostic apparatus having a function of evaluating the hardness of tissues in a subject has come into widespread use. There are two main methods for evaluating hardness using an ultrasound diagnostic apparatus. One method presses the tissues in the subject from the surface of the body, using an ultrasound probe, releases the pressure, and evaluates the relative hardness of the tissues in the subject from the amount of distortion of the tissues in the subject due to the pressure. This method can evaluate whether the tissues are harder or softer than peripheral tissues. That is, this method can evaluate the relative hardness of the tissues in the subject.

The other method generates shear waves in a region of interest (ROI) which is set in the subject, acquires the displacement of the tissues in the region of interest in time series, and evaluates the propagation speed of the shear waves. Since the propagation speed of the shear waves varies depending on the elastic modulus of the tissues, this method can evaluate the absolute hardness (for example, the elastic modulus) of the tissues. As a method for generating the shear waves, for example, a method is used which focuses a push pulse which is called an acoustic radiation force impulse (ARFI) and displaces the tissues in the subject on the focus, using the sound pressure of the push pulses which are ultrasonic waves. The use of the ARFI makes it possible to evaluate hardness even if the region of interest is so deep that it is not capable of being pressed from the surface of the body. Therefore, image diagnosis using both a so-called ultrasound image (B-mode image) and an elastic image obtained by evaluating hardness is performed.

However, when the ultrasound probe is moved during the evaluation of hardness, it is difficult to evaluate the hardness. For example, when the ultrasound probe is moved during image diagnosis using an ultrasound image and an elastic image which are generated at the same time, it may be difficult to determine whether a tissue image in the elastic image and a tissue image in the ultrasound image are the same tissue image. The reason is as follows. The transmission of push pulses and the propagation analysis of shear waves need to be performed in order to generate the elastic image. As a result, the frame rate of the elastic image is significantly lower than that of the ultrasound image. Therefore, when the ultrasound probe is moved, the deviation between the regions of interest in the elastic image and the ultrasound image occurs, which makes it difficult to perform diagnosis using the comparison between the tissue image in the elastic image and the tissue image in the ultrasound image which are related to the same tissue. In addition, for example, when the ultrasound probe is moved during a process of acquiring the displacement of the tissues due to the shear waves in time series, it is difficult to detect the displacement. JP 2013-544615 A discloses a technique that performs correction for excluding the influence of the movement of the ultrasound probe from the detected displacement. However, the correction is not necessarily sufficient.

### SUMMARY OF THE INVENTION

The present disclosure has been made in view of the above-mentioned problems and an object of the present disclosure is to provide an ultrasound diagnostic apparatus that can respond to the movement of an ultrasound probe.

To achieve the abovementioned object, according to an aspect, an ultrasound diagnostic apparatus that transmits a push pulse for focusing ultrasonic waves on a specific part of a subject to physically press tissues in the specific part using an ultrasound probe, repeatedly transmits and receives the ultrasonic waves to and from the subject using the ultrasound probe, and detects a propagation state of shear waves generated from the pressed tissues of the specific part, which are a vibration source, in a region of interest set in the subject, reflecting one aspect of the present invention comprises: a push pulse transmission unit that transmits the push pulse; a displacement detection unit that transmits detection waves to the subject a plurality of times after the push pulse is transmitted, receives reflected detection waves corresponding to the detection waves from the subject, generates a plurality of received signals in time series, and detects displacement of the tissues in the subject due to the shear waves caused by the push pulse at each time when the reflected detection waves are received; an elasticity measurement unit that analyzes the propagation state of the shear waves in the region of interest on the basis of a detection result of the displacement detection unit and measures elasticity of each tissue in the subject; a probe movement detection unit that detects a moving speed of the ultrasound probe; a sequence holding unit that holds a plurality of operation sequences defining a series of operations performed by the push pulse transmission unit, the displacement detection unit, and the elasticity measurement unit in cooperation with each other which enables the elasticity measurement unit to measure the elasticity; and a sequence selection unit that selects one operation sequence from the plurality of operation sequences held by the sequence holding unit on the basis of a detection result of the probe movement detection unit.

### BRIEF DESCRIPTION OF THE DRAWINGS

The above and other objects, advantages and features of the present invention will become more fully understood from the detailed description given hereinbelow and the appended drawings which are given by way of illustration only, and thus are not intended as a definition of the limits of the present invention, and wherein:

FIG. 1 is a block diagram illustrating an ultrasound diagnostic apparatus according to a first embodiment;

FIG. 2 is a flowchart illustrating the overall operation of the ultrasound diagnostic apparatus according to the first embodiment;

FIG. 3 is a flowchart illustrating a first operation sequence according to the first embodiment;

FIG. 4A is a diagram schematically illustrating the focal position of push pulses in the first operation sequence and FIG. 4B is a diagram schematically illustrating the focal position of the push pulses in a second operation sequence;

FIGS. 5A to 5E are diagrams schematically illustrating an aspect of the generation and propagation of shear waves;

FIGS. 6A to 6E are diagrams schematically illustrating a shear wave propagation analysis operation according to the first embodiment;

FIG. 7A is a diagram schematically illustrating the measurement of the speed of the shear waves and FIG. 7B is a diagram schematically illustrating an example of an elastic image;

FIGS. 8A-1 to 8A-4 are diagrams schematically illustrating the analysis results of propagation for each sub-sequence in the first operation sequence, FIG. 8B is a diagram schematically illustrating the analysis results of propagation for each sub-sequence, FIG. 8C is a diagram schematically illustrating an example of a weighting coefficient in the integration of the analysis results of propagation, and FIGS. 8D-1 and 8D-2 are diagrams schematically illustrating the analysis results of propagation for each sub-sequence in the second operation sequence;

FIG. 9 is a flowchart illustrating the second operation sequence according to the first embodiment;

FIG. 10 is a flowchart illustrating the overall operation of an ultrasound diagnostic apparatus according to a second embodiment;

FIG. 11 is a flowchart illustrating a third operation sequence according to the second embodiment;

FIGS. 12A-1 and 12A-2 are diagrams schematically illustrating the transmission of detection waves when the detection waves are plane waves, FIGS. 12B-1 and 12B-2 are diagrams schematically the transmission of the detection waves when the detection waves are focus waves, and FIGS. 12C-1 and 12C-2 are schematic diagrams when reflected detection waves are intermittently received in terms of time;

FIG. 13 is a flowchart illustrating a third operation sequence according to a modification example of the second embodiment;

FIG. 14 is a flowchart illustrating the overall operation of an ultrasound diagnostic apparatus according to a third embodiment;

FIG. 15 is a flowchart illustrating a fourth operation sequence according to the third embodiment;

FIGS. 16A and 16B are schematic diagrams when the regions of interest in a tomographic image signal and a reference tomographic image signal are not matched with each other, FIG. 16C is a diagram schematically illustrating a displacement detection operation in the first operation sequence, and FIG. 16D is a diagram schematically illustrating a displacement detection operation in the fourth operation sequence;

FIG. 17 is a flowchart illustrating the overall operation of an ultrasound diagnostic apparatus according to a fourth embodiment;

FIG. 18 is a flowchart illustrating a fifth operation sequence according to the fourth embodiment; and

FIG. 19 is a flowchart illustrating the overall operation of an ultrasound diagnostic apparatus according to another modification example (1).

### DESCRIPTION OF THE PREFERRED EMBODIMENTS

Hereinafter, an embodiment of the present invention will be described with reference to the drawings. However, the scope of the invention is not limited to the illustrated examples.

### How Embodiments of the Invention were Achieved

The inventors conducted various examinations in an ultrasound diagnostic apparatus that evaluated the hardness of tissues using shear waves in order to respond to the movement of an ultrasound probe.

In the ultrasound diagnostic apparatus that evaluates the hardness of the tissues using the shear waves, there are a plurality of influences of the movement of the ultrasound probe. Therefore, problems caused by each influence will be described.

First, in the ultrasound diagnostic apparatus that evaluates the hardness of the tissues, in general, an elastic image in which the distribution of hardness in a region of interest is represented by colors is displayed or the elastic image is displayed so as to be superimposed on an ultrasound image having the same region of interest as the elastic image. Here, the ultrasound image is, for example, a B-mode tomographic image. The reason is as follows. When the shape of a tissue having a different hardness from peripheral tissues is visually displayed and the hardness of the tissue is represented by colors, the operator more easily recognizes the tissue than that when the hardness of tissues at each position of the region of interest is represented by values. In addition, since the elastic image is compared with the ultrasound image having the same region of interest as the elastic image, the operator can know the image of the tissue in the ultrasound image and this method assists in diagnosis. In contrast, as described above, since the time required to transmit push pulses and to perform propagation analysis of shear wave is long, the frame rate of the elastic image is significantly lower than the frame rate of the ultrasound image. Therefore, when the ultrasound probe is moved at a certain speed or more, the regions of interest in the elastic image and the ultrasound image are not matched with each other and it is difficult to exactly associate the tissue images. Hereinafter, the reason will be described. In each of the ultrasound image and the elastic image, when the ultrasound probe is moved for the period from the start to the end of the generation of an image, the actual region of the subject in which ultrasonic waves are transmitted and received, that is, a region which is processed as the region of interest by the ultrasound diagnostic apparatus is not matched with a region from which the operator acquires information, that is, an examination target region assumed by the operator. The reason is as follows. The ultrasound diagnostic apparatus sets the region of interest as a relative position based on the position and direction of the ultrasound probe. Therefore, when the position or direction of the ultrasound probe is changed, the region of interest is moved in operative association with the change in the position or direction. When the moving speed of the ultrasound probe is constant, the amount of movement of the ultrasound probe between frames increases as the frame rate is reduced. Therefore, as the frame rate is reduced, the amount of movement of the region of interest increases. For this reason, even if the operator sets the region of interest such that the examination target regions are the same in the ultrasound image and the elastic image, the amounts of movement of the region of interest are different in the ultrasound image and the elastic image and the regions of interest in the ultrasound image and the elastic image are not matched with each other. Therefore, a coordinate point in the elastic image and a coordinate point in the ultrasound image which is the same as the coordinate point in the elastic image do not necessarily correspond to the same position in the subject. As a result, the tissue image in the elastic image and the tissue image in the ultrasound image, which have been acquired from the same tissue, are not capable of being associated with each other.

The inventors conducted an examination on a technique for improving the frame rate of an elastic image, considering the above-mentioned problems.

In addition, the ultrasound diagnostic apparatus that evaluates the hardness of the tissues detects displacement in the region of interest. As a method for detecting displacement, the following methods are used: a method that repeatedly transmits and receives ultrasonic waves to acquire received signals in time series while shear waves are propagated and detects displacement on the basis of the absolute difference between each acquired received signal and a reference signal which is acquired at the time when no displacement occurs; and a method that detects displacement on the basis of the relative difference (the time rate of change in the absolute difference) between each received signal acquired in time series and the reference signal. In the method based on the relative difference, even if the ultrasound probe is moved, the overlap area between the regions of interest in the received signals is not very small and it is easy to detect displacement. However, errors in the relative differences are accumulated and the accuracy of the amount of displacement is likely to be reduced. In contrast, in the method based on the absolute difference, it is easy to improve the accuracy of the amount of displacement. However, when the regions of interest in the received signal and the reference signal deviate from each other, the size of a region in which displacement is not capable of being calculated increases as the deviation between the regions of interest increases. In the related art, displacement is detected on the basis of the absolute difference on the assumption that the ultrasound probe is not moved while the hardness of tissues is being evaluated. When the moving speed of the ultrasound probe is high, the region in which displacement is not capable of being calculated region is extended. In some cases, a region in which the elasticity of tissues is not capable of being evaluated is generated.

The inventors conducted an examination on a technique for changing a displacement calculation method according to the moving speed of the ultrasound probe, considering the above-mentioned problems.

The inventors conducted an examination on a technique that did not start to generate an elastic image until the moving speed of the ultrasound probe was less than a predetermined speed when the moving speed of the ultrasound probe was greater than the predetermined speed, from the point of view that a high moving speed of the ultrasound probe caused the above-mentioned various problems and was not suitable for evaluating the hardness of tissues.

The inventors conceived a technique for changing a portion of an operation for evaluating the hardness of tissues according to the moving speed of an ultrasound probe, on the basis of the examination result, and ultrasound diagnostic apparatuses according to embodiments were achieved.

Hereinafter, ultrasound diagnostic apparatuses according to the embodiments will be described in detail with reference to the drawings.

### First Embodiment

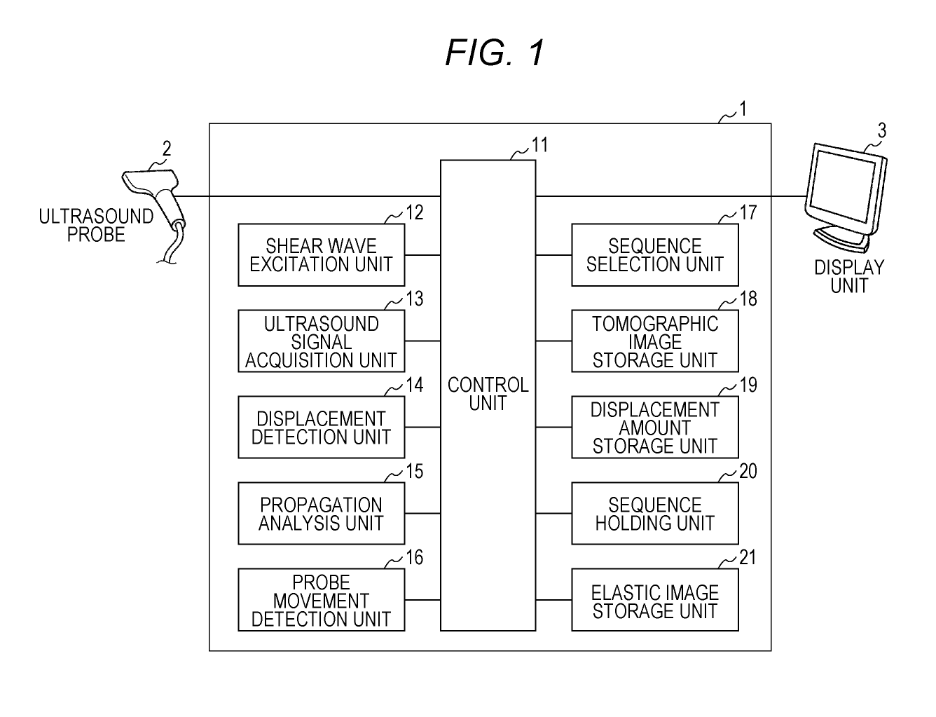

FIG. 1 is a block diagram illustrating an ultrasound diagnostic apparatus 1 according to the first embodiment. The ultrasound diagnostic apparatus 1 includes a control unit 11, a shear wave excitation unit 12, an ultrasound signal acquisition unit 13, a displacement detection unit 14, a propagation analysis unit 15, a probe movement detection unit 16, a sequence selection unit 17, a tomographic image storage unit 18, a displacement amount storage unit 19, a sequence holding unit 20, and an elastic image storage unit 21. An ultrasound probe 2 and a display unit 3 are configured so as to be connected to the control unit 11. FIG. 1 illustrates a state in which the ultrasound probe 2 and the display unit 3 are connected to the ultrasound diagnostic apparatus 1.

The ultrasound probe 2 includes, for example, a plurality of transducers (not illustrated) which are arranged in a one-dimensional direction. Each transducer is made of, for example, lead zirconate titanate (PZT). The ultrasound probe 2 receives an electric signal (hereinafter, referred to as an “ARFI driving signal”) generated by the shear wave excitation unit 12 or an electric signal (hereinafter, referred to as a “transmission driving signal”) generated by the ultrasound signal acquisition unit 13 from the control unit 11 and converts the electric signal into ultrasonic waves. The ultrasound probe 2 transmits an ultrasonic wave beam including a plurality of ultrasonic waves, which have been converted from the ARFI driving signal or the transmission driving signal and emitted from a plurality of transducers, to a measurement target of a subject in a state in which a transducer-side outer surface of the ultrasound probe 2 is in contact with a surface such as the surface of the skin of the subject. Then, the ultrasound probe 2 receives a plurality of reflected detection waves, which correspond to transmitted detection waves based on the transmission driving signal, from the measurement target, converts the reflected detection waves into electric signals (hereinafter, referred to as “element reception signals”) using a plurality of transducers, and supplies element reception signals to the ultrasound signal acquisition unit 13 through the control unit 11.

The shear wave excitation unit 12 generates the ARFI driving signal which is an electric signal for transmitting push pulses to the ultrasound probe 2. The push pulse is a pulsed ultrasonic wave for displacing tissues in the subject in order to generate shear waves in the subject. Specifically, the push pulse is an ultrasonic wave that is focused on a certain region in a region of interest in the subject and has a larger wave number than a transmitted detection wave which will be described below. Therefore, the ARFI driving signal is a pulsed electric signal that is generated such that ultrasonic waves generated from each transducer element of the ultrasound probe 2 are transmitted to the focus. The shear wave excitation unit 12 receives, for example, the focal position of each push pulse, the transducer used for transmission, a wave number, or transmission duration defined in an operation sequence, which will be described below, from the control unit 11 and generates the ARFI driving signal on the basis of the operation sequence.

The ultrasound signal acquisition unit 13 transmits the transmission driving signal which is an electric signal for transmitting the transmitted detection wave to the ultrasound probe 2. For example, the transmission driving signal is a pulsed electric signal that is generated such that the transmitted detection waves transmitted from each transducer element of the ultrasound probe 2 become plane waves which travel in a specific direction and synchronizes the operations of each transducer element or shifts the operation time stepwise from one end to the other end of a transducer column at a fixed pitch. Alternatively, the transmission driving signal may be, for example, a pulsed electric signal that is generated such that the transmitted detection waves transmitted from each transducer element of the ultrasound probe 2 become focus waves which reach a transmission focus point at the same time and makes the transducer elements operate at different times. In addition, the ultrasound signal acquisition unit 13 performs phasing addition for the element reception signal based on the reflected detection waves to generate an acoustic line signal. When the transmitted detection waves are plane waves, the transmitted detection waves are transmitted so as to pass through the entire region of interest and an acoustic line signal for the entire region of interest is generated on the basis of reflected ultrasonic waves. In contrast, when the transmitted detection waves are focus waves, an acoustic line signal based on the reflected ultrasonic waves is generated for each region, which is obtained by dividing the region of interest including a transmission focus point and the periphery of the transmission focus point in a portion of the region through which the transmitted detection waves have passed, in the direction of an element column. Therefore, when the transmitted detection waves are focus waves, the transmission of the transmitted detection waves and the reception of the reflected detection waves are repeatedly performed while the transmission focus point is moved in the direction of the element column, in order to obtain the acoustic line signal for the entire region of interest. The ultrasound signal acquisition unit 13 outputs the generated acoustic line signal to the tomographic image storage unit 18 through the control unit 11.

The displacement detection unit 14 acquires a plurality of acoustic line signals (hereinafter, referred to as “tomographic image signals”) related to one tomographic image which is a displacement detection target and a plurality of reference acoustic line signals (hereinafter, referred to as “reference tomographic image signals”) related to one tomographic image from the tomographic image storage unit 18 through the control unit 11. The reference tomographic image signal is used to extract displacement due to shear waves from the tomographic image signal. Specifically, the reference tomographic image signal is a tomographic image signal obtained by capturing the region of interest before a push pulse is transmitted. Then, the displacement detection unit 14 detects the displacement of each pixel of the tomographic image signal from the difference between the tomographic image signal and the reference tomographic image signal and generates a displacement image in which displacement is associated with the coordinates of each pixel. The displacement detection unit 14 outputs the generated displacement image to the displacement amount storage unit 19 through the control unit 11.

The propagation analysis unit 15 acquires the displacement image from the displacement amount storage unit 19 through the control unit 11. The propagation analysis unit 15 detects the position of the wave front of the shear waves and the traveling direction and speed of the shear waves at each time when the displacement image is acquired from the displacement image, calculates the elastic modulus of the tissues of the subject corresponding to each pixel of the displacement image, and generates an elastic image. The propagation analysis unit 15 outputs the generated elastic image to the elastic image storage unit 21 through the control unit 11.

The probe movement detection unit 16 detects the moving speed of the ultrasound probe 2 and outputs the moving speed to the sequence selection unit 17. Specifically, the probe movement detection unit 16 acquires the latest tomographic image signal and a previous tomographic image signal from the tomographic image storage unit 18 and detects the moving speed of the ultrasound probe 2 from the difference between the two tomographic image signals. For example, the moving speed of the ultrasound probe 2 can be calculated by calculating the difference (displacement) between the latest tomographic image signal and the previous tomographic image signal for each pixel and multiplying the minimum value of the difference for each pixel by the frame rate of the tomographic image. In addition, other representative values, such as an intermediate value, may be used, instead of the minimum value of the difference for each pixel. For example, the moving speed of the ultrasound probe 2 may be calculated using the difference for the pixels corresponding to the positions through which the shear waves are not capable of being passing, such as the focal positions of push pulses and the positions where only the depth is different. Alternatively, when the difference (displacement) is calculated, only the difference (displacement) between components in the arrangement direction of the elements may be detected. The reason is that, since displacement caused by the shear waves occurs in the depth direction in principle, the difference (displacement) in the arrangement direction of the elements is likely to be caused by the movement of the ultrasound probe 2. A method for detecting the moving speed of the ultrasound probe 2 is not limited to the above-mentioned method and any method using a tomographic image signal may be used. Alternatively, for example, the ultrasound probe 2 may further include a speed sensor and the probe movement detection unit 16 may use the detection value of the speed sensor. Alternatively, for example, the ultrasound diagnostic apparatus 1 may further include a camera for detecting the movement of the ultrasound probe 2, the ultrasound probe 2 may include a mark for detecting the position and direction of the ultrasound probe 2 using the camera, and the probe movement detection unit 16 may detect the movement of the marker from an image acquired by the camera ultrasound probe 2 to detect the moving speed.

The sequence selection unit 17 selects one operation sequence from a plurality of operation sequences held by the sequence holding unit 20, using the moving speed of the ultrasound probe 2 detected by the probe movement detection unit 16. Here, the operation sequence indicates a series of operations of the ultrasound diagnostic apparatus 1 generating one elastic image. Specifically, the operation sequence includes at least one or more push pulse transmission operations, an operation for transmitting detection waves and receiving reflected detection waves for each push pulse transmission operation, and a propagation analysis operation. That is, a series of operations performed by the shear wave excitation unit 12, the ultrasound signal acquisition unit 13, the displacement detection unit 14, and the propagation analysis unit 15 in cooperation with each other is assumed the operation sequence. The operation sequence includes, for example, the number of times a push pulse is transmitted, the focal position of each push pulse, the transducer used for transmission, a wave number, transmission duration, information indicating whether detection waves are plane waves or focus waves, the transmission direction of detection waves when the detection waves are plane waves, the number of transmission focus points, the position of each transmission focus point, and an acoustic line signal generation region corresponding to the position of each transmission focus point when detection waves are focus waves, and the frame rate of a tomographic image signal. In addition, the operation sequence may further include information for defining operations associated with a series of operations, such as the detection wave transmission operation, the reflected detection wave receiving operation, and the propagation analysis operation. For example, the operation sequence may include information indicating whether to generate an elastic image and information about the display form of an elastic image.

The control unit 11 performs an operation of outputting the tomographic image generated by the ultrasound signal acquisition unit 13 and the elastic image generated by the propagation analysis unit 15 to the display unit 3, in addition to an operation of controlling each of the above-mentioned components. When the elastic image is output to the display unit 3, the control unit 11 performs geometric transformation. When the tomographic image is output to the display unit 3, the control unit 11 performs, for example, envelope detection and logarithmic compression, in addition to geometric transformation.

The tomographic image storage unit 18, the displacement amount storage unit 19, the sequence holding unit 20, and the elastic image storage unit 21 store tomographic images, displacement images, operation sequence data, and elastic image data, respectively. Each of the tomographic image storage unit 18, the displacement amount storage unit 19, the sequence holding unit 20, and the elastic image storage unit 21 is implemented by a storage medium, such as a RAM, a flash memory, a hard disk, or an optical disk. Two or more of the tomographic image storage unit 18, the displacement amount storage unit 19, the sequence holding unit 20, and the elastic image storage unit 21 may be implemented by a single storage medium. Alternatively, the tomographic image storage unit 18, the displacement amount storage unit 19, the sequence holding unit 20, and the elastic image storage unit 21 may be implemented in other components of the ultrasound diagnostic apparatus 1. For example, the sequence holding unit 20 may be a portion of the sequence selection unit 17. In addition, one or more of the tomographic image storage unit 18, the displacement amount storage unit 19, the sequence holding unit 20, and the elastic image storage unit 21 may be provided outside the ultrasound diagnostic apparatus 1 and may be connected to the ultrasound diagnostic apparatus 1 through an interface, such as a USB, eSATA, or SDIO, or may be resources which can be accessed by the ultrasound diagnostic apparatus 1 through a network, for example, a file server or a network attached storage (NAS).

Each of the control unit 11, the shear wave excitation unit 12, the ultrasound signal acquisition unit 13, the displacement detection unit 14, the propagation analysis unit 15, the probe movement detection unit 16, and the sequence selection unit 17 may be implemented by hardware, such as a field programmable gate array (FPGA) or an application specific integrated circuit (ASIC). Two or more of them may be configured as a single component. For example, the shear wave excitation unit 12 and the ultrasound signal acquisition unit 13 may be configured as one component. In this case, the ARFI driving signal is generated using the same structure as that for generating the transmission driving signal of the ultrasound signal acquisition unit 13, which makes it possible to implement the shear wave excitation unit 12 using the structure of the ultrasound signal acquisition unit 13. Some or all of them may be implemented by a single FPGA or ASIC. In addition, each of the above-mentioned units or each unit including two or more of the above-mentioned units may be implemented by a memory, a programmable device, such as a central processing unit (CPU), or a general purpose graphic processing unit (GPGPU), and software.

<Operation>

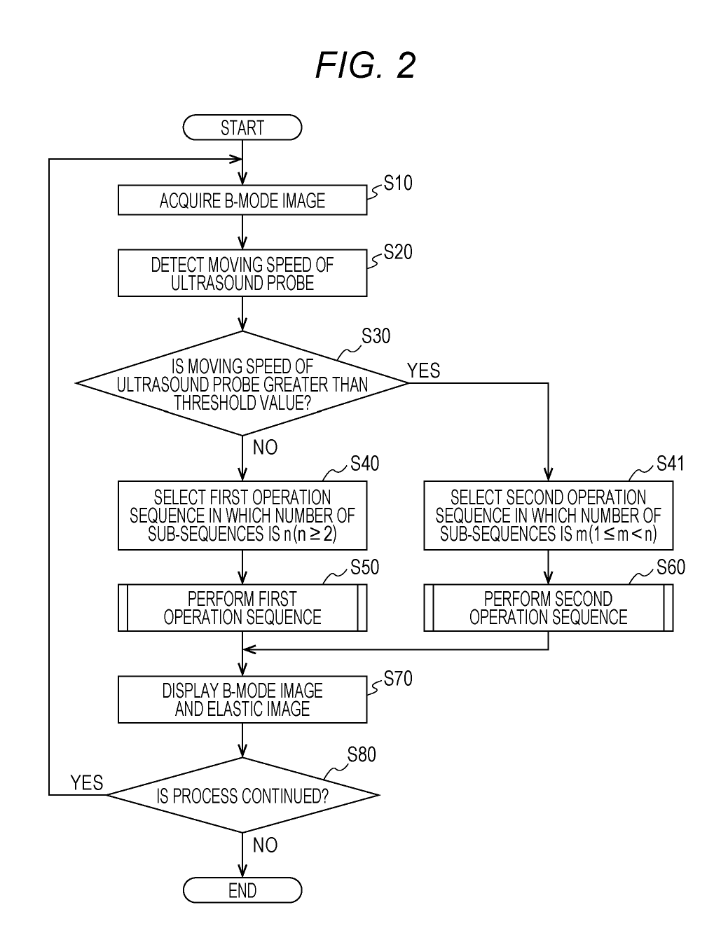

The operation of the ultrasound diagnostic apparatus 1 according to the first embodiment will be described. FIG. 2 is a flowchart illustrating the overall operation of the ultrasound diagnostic apparatus 1.

First, ultrasonic waves are transmitted to and received from the subject and an acquired received signal is stored (step S10). Specifically, the following operation is performed. First, a transmission event is performed as follows. At the beginning, the ultrasound signal acquisition unit 13 generates a pulsed transmission signal. Then, the ultrasound signal acquisition unit 13 forms a transmission beam for setting the delay time of each element of the ultrasound probe 2 for the transmission signal and generates a plurality of transmission driving signals corresponding to each element of the ultrasound probe 2. Each transducer of the ultrasound probe 2 converts the corresponding transmission driving signal into ultrasonic waves and an ultrasonic beam is transmitted to the subject. Then, each transducer of the ultrasound probe 2 acquires ultrasonic waves reflected from the subject and converts the reflected ultrasonic waves into an element reception signal. The ultrasound signal acquisition unit 13 performs phasing addition for the element reception signal to generate an acoustic line signal. The control unit 11 acquires the acoustic line signal from the ultrasound signal acquisition unit 13 for each transmission event and stores a plurality of acoustic line signals forming one tomographic image in the tomographic image storage unit 18.

Then, the moving speed of the ultrasound probe 2 is detected (step S20). Specifically, the probe movement detection unit 16 acquires an acoustic line signal related to the latest transmission event and an acoustic line signal related to the previous transmission event from the tomographic image storage unit 18 and detects the difference (displacement) between the acoustic line signals using a correlation process. For example, the probe movement detection unit 16 can multiply a representative value, such as the minimum value or intermediate value of the difference, by a difference in the execution time of the transmission event to calculate the moving speed of the ultrasound probe 2.

Next, an operation sequence is determined (steps S30 and S40). Specifically, the sequence selection unit 17 determines whether the moving speed of the ultrasound probe 2 is greater than a predetermined threshold value (step S30). Here, the predetermined threshold value is, for example, 10 mm/s. When the moving speed of the ultrasound probe 2 is equal to or less than the predetermined threshold value (No in S30), the sequence selection unit 17 selects a first operation sequence that is the same as that of the ultrasound diagnostic apparatus according to the related art (step S40). In the first operation sequence, the region of interest is divided into n (n is an integer equal to or greater than 2) small regions and one push pulse transmission operation and a plurality of detection wave transmitting and receiving operations which follow the push pulse transmission operation are performed for each small region to perform shear wave propagation analysis (a series of operations is referred to as a “sub-sequence”). Then, the analysis results of propagation by n sub-sequences are combined with each other to generate an elastic image. Hereinafter, a case in which n is 4 will be described. On the other hand, when the moving speed of the ultrasound probe 2 is greater than the predetermined threshold value (Yes in S30), the sequence selection unit 17 selects a second operation sequence (step S41). In the second operation sequence, in order to improve the frame rate of the elastic image, the region of interest is divided into m (m is an integer that is equal to or greater than 1 and is less than n) small regions, propagation analysis is performed for each small region by the sub-sequence, and the analysis results of propagation by m sub-sequences are combined with each other to generate an elastic image. Hereinafter, a case in which m is 2 will be described.

Then, the operation sequence is performed (steps S50 and S60).

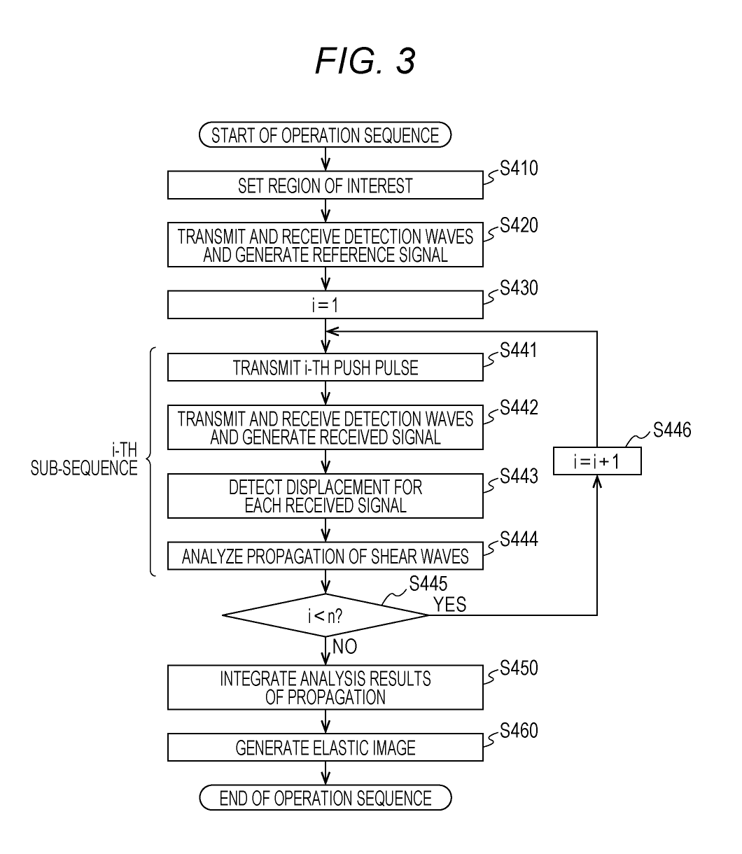

Here, only step S50 will be described. The difference between step S60 and step S50 will be described below. FIG. 3 is a flowchart illustrating the detailed operation of the operation sequence in S50.

First, the control unit 11 sets a region of interest (step S410). For example, a method for setting the region of interest is as follows. The latest tomographic image stored in the tomographic image storage unit 18 is displayed on the display unit 3 such that the operator designates the region of interest through an input unit (not illustrated), such as a touch panel, a mouse, or a trackball. The method for setting the region of interest is not limited thereto. For example, the entire tomographic image may be set as the region of interest or a certain range including a central portion of the tomographic image may be set as the region of interest. In addition, when the region of interest is set, another tomographic image may be acquired.

Then, ultrasonic waves are transmitted to and received from the region of interest and an acquired received signal is stored as the reference signal (step S420). Specifically, a transmission event is performed and a plurality of acoustic line signals forming one tomographic image are stored as the reference tomographic image signals in the tomographic image storage unit 18.

Then, a sub-sequence including a push pulse transmission operation, a plurality of detection wave transmitting and receiving operations following the push pulse transmission operation, and a shear wave propagation analysis operation is performed. First, a first sub-sequence (steps S441 to S444 when i is 1) is performed (step S430).

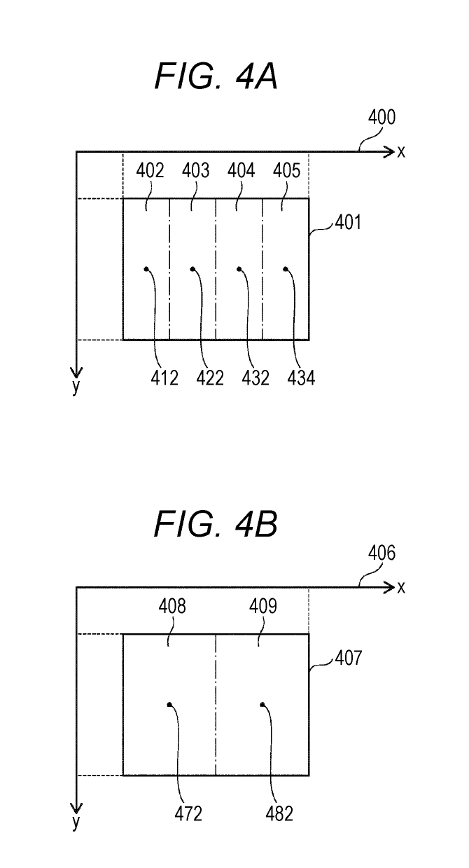

In the first sub-sequence, first, a push pulse is transmitted (step S441). Specifically, the shear wave excitation unit 12 generates a pulsed ARFI signal on the basis of the focal position of a push pulse which is defined as a first push pulse in the first operation sequence, a transducer used for transmission, a wave number, or transmission duration. Then, the shear wave excitation unit 12 forms a transmission beam for setting the delay time of each element of the ultrasound probe 2 for the ARFI signal and generates a plurality of ARFI driving signals corresponding to each element of the ultrasound probe 2. The focal position of the first push pulse is, for example, the center of one of n small regions (here, n is 4) obtained by dividing the region of interest in the column direction of the transducers. A detailed example will be described with reference to FIG. 4A. In FIG. 4A, the x direction is the column direction of the transducers and the y direction is a depth direction. In this case, a region of interest 401 is divided into four small regions 402, 403, 404, and 405 and the first push pulse is transmitted so as to be focused on a focal position 412 in the small region 402. Each transducer of the ultrasound probe 2 converts the corresponding transmission driving signal into ultrasonic waves and push pulses are transmitted into the subject.

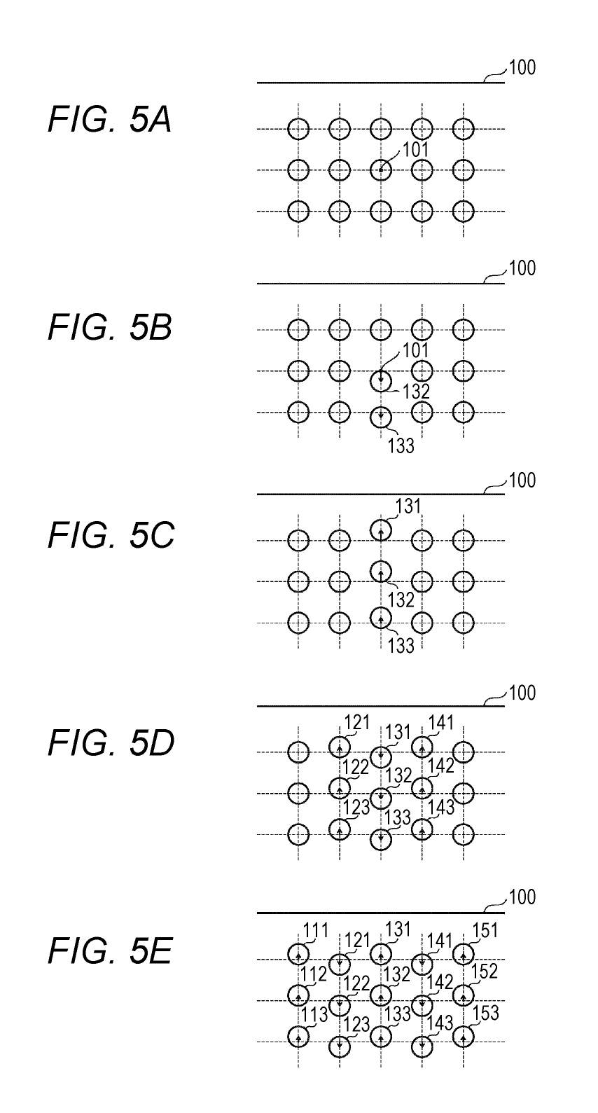

Here, the generation of shear waves by push pulses will be described with reference to the schematic diagrams illustrated in FIGS. 5A to 5E. FIG. 5A is a diagram schematically illustrating tissues in a region of the subject which corresponds to the region of interest before push pulses are applied. In FIGS. 5A to 5E, “o” indicates some of the tissues of the subject in the region of interest and an intersection point between dashed lines indicates the center of the tissue “o” when there is no load. Here, when push pulses are applied to a focus 101 with the ultrasound probe 2 coming into close contact with a skin surface 100, a tissue 132 that is located at the focus 101 is pushed and moved in the traveling direction of the push pulses, as illustrated in the schematic diagram of FIG. 5B. In addition, a tissue 133 which is close to the tissue 132 in the traveling direction of the push pulses is pushed to the tissue 132 and is moved in the traveling direction of the push pulses. Then, when the transmission of the push pulses ends, the tissues 132 and 133 return to the original positions. Therefore, as illustrated in the schematic diagram of FIG. 5C, the tissues 131 to 133 start to vibrate in the traveling direction of the push pulses. Then, as illustrated in the schematic diagram of FIG. 5D, vibration is propagated to tissues 121 to 123 and tissues 141 to 143 which are adjacent to the tissues 131 to 133. In addition, as illustrated in the schematic diagram of FIG. 5E, vibration is further propagated to tissues 111 to 113 and tissues 151 to 153. Therefore, in the subject, vibration is propagated in a direction operation the vibration direction. That is, shear waves are generated at the position where the push pulses are applied and are propagated through the subject.

Returning to FIG. 3, the description will be continued. Then, ultrasonic waves are transmitted to and received from the region of interest a plurality of times and a plurality of acquired ultrasound signals are stored (step S442). Specifically, immediately after the transmission of the push pulses ends, the same operation as that in step S10 is repeated, for example, 10000 times per second. In this way, the tomographic image of the subject is repeatedly acquired until propagation ends immediately after shear waves are generated.

Then, the displacement of each pixel is detected (step S443). Specifically, first, the displacement detection unit 14 acquires the reference tomographic image signal stored in the tomographic image storage unit 18 in step S420. Then, the displacement detection unit 14 detects the displacement of each pixel at the time when the reflected detection waves related to each tomographic image signal stored in the tomographic image storage unit 18 in step S442 are received from the difference between the reference tomographic image signal and the tomographic image signal. Specifically, for example, a correlation process between the tomographic image signal and the reference tomographic image signal is performed to search for the correspondence relationship between the pixels of the tomographic image signal and the pixels of the reference tomographic image signal and the difference between the coordinates of the pixels is specified as displacement corresponding to the pixels of the tomographic image signal. A method for detecting the displacement is not limited thereto the correlation process. For example, any technique for detecting the amount of movement between two tomographic image signals, such as pattern matching, may be used. An example of the pattern matching is a method that divides a tomographic image signal is regions with a predetermined size, such as a size of 8 pixels×8 pixels and performs pattern matching between each region and the reference tomographic image signal to detect the displacement of each pixel of the tomographic image signal. An example of the pattern matching method is a method that calculates the difference between the brightness values of the corresponding pixels in each region and a reference region which has the same size as the region in the reference tomographic image signal, calculates the sum of the absolute values of the differences, considers a region and the reference region, which form a pair and have the minimum sum, to be the same region, and detects the distance between a reference point (for example, the upper left corner) of the region and a reference point of the reference region as the displacement. In addition, the region with the predetermined size may have other sizes and, for example, the sum of the squares of the differences between the brightness values may be used, instead of the sum of the absolute values of the differences between the brightness values. When displacement is detected by the correlation process or the pattern matching, the difference (difference in depth) between the y-coordinates of the corresponding pixels may be used as the magnitude of displacement, in addition to the difference between the coordinates of the corresponding pixels. The reason is that, since the propagation direction of shear waves is the column direction (x direction) of the elements in principle, the direction of the displacement caused by the shear waves is a direction perpendicular to the propagation direction and is the depth direction (y direction) in principle. The amount of movement of the tissues of the subject, which correspond to each pixel of each tomographic image signal, by the push pulses or the shear waves is calculated as displacement by the above-mentioned process. The displacement detection unit 14 associates the displacement of each pixel of one tomographic image with the coordinates of the pixels to generate a displacement image and outputs the generated displacement image to the displacement amount storage unit 19.

Then, shear wave propagation analysis is performed (step S444). Specifically, the wave front of the shear waves is extracted from each displacement image and a wave front image is generated. The use of the wave front image makes it possible to easily detect the position, amplitude, traveling direction, and speed of the wave front. The wave front image is generated by, for example, the order of a displacement region extraction process, a thinning process, a spatial filtering process, and a temporal filtering process.

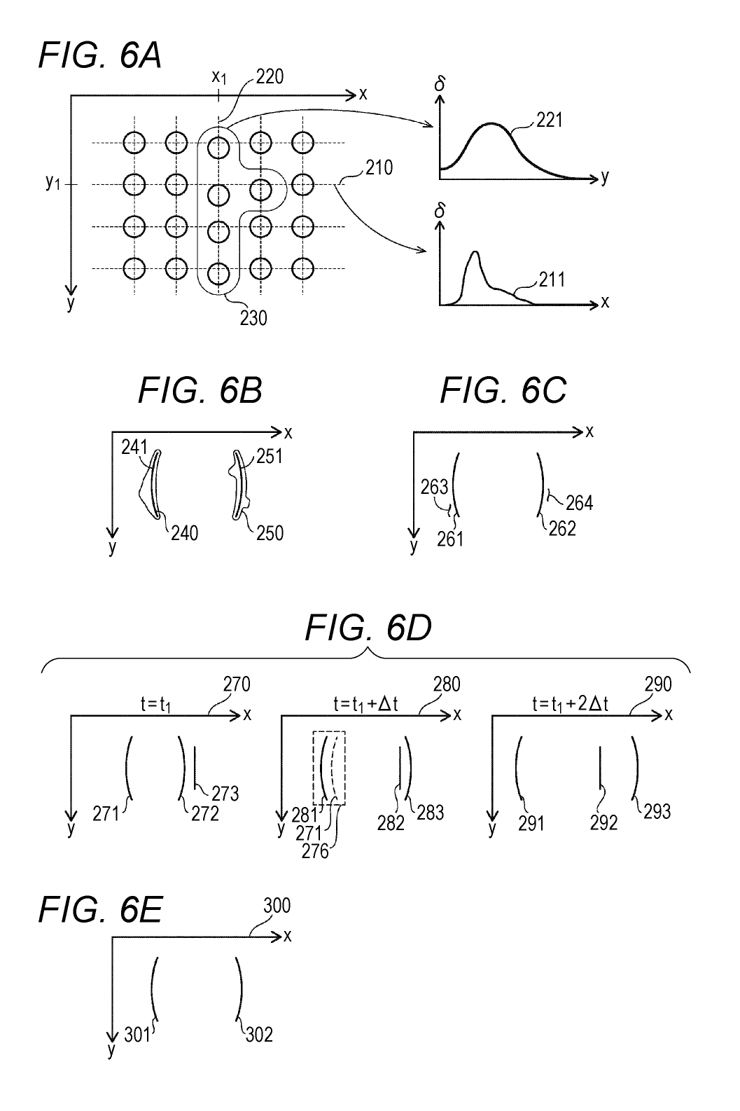

A detailed process will be described with reference to FIGS. 6A to 6E. FIG. 6A illustrates an example of the displacement image. Similarly to FIGS. 5A to 5E, in FIG. 6A, “o” indicates some of the tissues of the subject in the region of interest and an intersection point between dashed lines indicates a position before push pulses are applied. The propagation analysis unit 15 extracts a region with a large displacement amount δ on the basis of a dynamic threshold value, using the displacement amount δ for each y-coordinate as the function of a coordinate x. In addition, the propagation analysis unit 15 extracts a region that is greater than a certain threshold value as the region with a large displacement amount δ on the basis of the dynamic threshold value, using the displacement amount δ for each x-coordinate as the function of a coordinate y. The dynamic threshold value is a threshold value determined by performing signal analysis or image analysis for a target region. The threshold value is not a constant value and varies depending on, for example, the width or maximum value of the signal of the target region. FIG. 6A illustrates a graph 211 obtained by plotting a displacement amount on a straight line 210 corresponding to y=y₁ and a graph 221 obtained by plotting a displacement amount on a straight line 220 corresponding to x=x₁. In this way, for example, it is possible to extract a displacement region 230 in which the displacement amount δ is greater than the threshold value.

Then, the propagation analysis unit 15 performs a thinning process for the displacement region to extract the wave front. Displacement regions 240 and 250 illustrated in the schematic diagram of FIG. 6B are the regions that have been extracted as the displacement regions in step S52. The propagation analysis unit 15 extracts the wave front, using, for example, a Hilditch thinning algorithm. For example, in the schematic diagram of FIG. 6B, a wave front 241 is extracted from the displacement region 240 and a wave front 251 is extracted from the displacement region 250. The thinning algorithm is not limited to the Hilditch thinning algorithm and any thinning algorithm may be used. A process that removes the coordinates where the displacement amount δ is equal to or less than the threshold value from the displacement region is repeatedly performed for each displacement region until the displacement region becomes a line with a width of one pixel, while increasing the threshold value.

Then, the propagation analysis unit 15 performs spatial filtering for wave front image data subjected to the thinning process to remove a wave front with a small length. For example, the propagation analysis unit 15 detects the length of each wave front extracted in step S53 and removes a wave front with a length that is less than half of the average value of the lengths of all of the wave fronts as noise. Specifically, as illustrated in the wave front image of FIG. 6C, the propagation analysis unit 15 calculates the average value of the lengths of wave fronts 261 to 264 and removes the wave fronts 263 and 264 with a length that is less than the average value as noise. In this way, it is possible to remove a wave front that has been erroneously detected.

The propagation analysis unit 15 performs the displacement region extraction process, the thinning process, and the spatial filtering operation for all of the displacement images. In this way, wave front image data which is in one-to-one correspondence with the displacement images is generated.

Finally, the propagation analysis unit 15 performs temporal filtering for a plurality of wave front image data items to remove the wave front which has not been propagated. Specifically, the propagation analysis unit 15 detects a change in the position of the wave front over time in two or more temporally continuous wave front images and removes a wave front of which the speed is abnormal as noise. For example, the propagation analysis unit 15 detects a change in the position of the wave front over time in a wave front image 270 at a time t=t₁, a wave front image 280 at a time t=t₁+Δt, and a wave front image 290 at a time t=t₁+2Δt. For example, the propagation analysis unit 15 performs a correlation process with a wave front 271 in a region 276, in which a shear wave is moved from the same position as that of the wave front 271 for a time Δt in a direction (the x-axis direction in FIGS. 6A to 6E) perpendicular to the wave front, in the wave front image 280. In this case, the correlation process is performed in a range including both the positive direction (the right side of FIGS. 6A to 6E) and the negative direction (the left side of FIGS. 6A to 6E) of the x-axis of the wave front 271, in order to detect both transmitted waves and reflected waves. In this way, it is detected that the movement destination of the wave front 271 is a wave front 281 in the wave front image 280 and the moving distance of the wave front 271 for the time Δt is calculated. Similarly, for each of wave fronts 272 and 273, the propagation analysis unit 15 performs the correlation process with each of the wave fronts in a region, in which a shear wave is moved from the same position as that of the wave fronts for the time Δt in a direction perpendicular to the wave fronts, in the wave front image 280. In this way, it is detected that the wave front 272 has moved to the position of the wave front 283 and the wave front 273 has moved to the position of the wave front 282. The same process is performed for the wave front image 280 and the wave front image 290 and it is detected that the wave front 281 has moved to the position of a wave front 291, the wave front 282 has moved to the position of a wave front 292, and the wave front 283 has moved to the position of a wave front 293. Here, the moving distance of one wave front represented as the wave front 273, the wave front 282, or the wave front 292 is significantly less than that of other wave fronts (the propagation speed thereof is significantly lower than that of other wave fronts). This wave front is regarded as noise and is removed since it is likely to be erroneously detected. In this way, it is possible to detect wave fronts 301 and 302 as illustrated in a wave front image 300 of FIG. 6E.

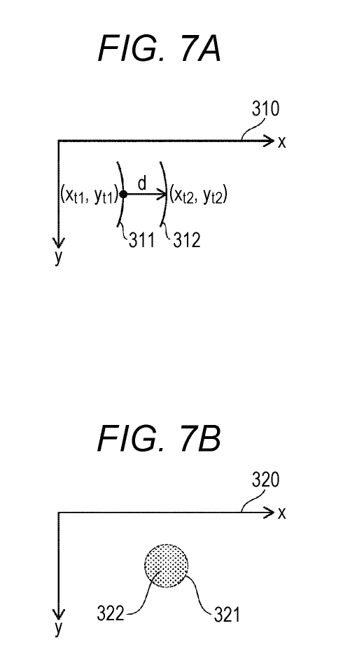

The propagation analysis unit 15 calculates the position and speed of the wave front, using the generated wave front image data at each time and the correspondence information of the wave fronts. Here, the correspondence information of the wave fronts indicates information about the correspondence between the wave fronts considered as the same wave front in the wave front images. For example, in FIG. 6D, when it is detected that the wave front 272 has moved to the position of the wave front 283, the correspondence information of the wave front is information indicating that the wave front 283 and the wave front 272 are the same wave front. Next, a method for calculating the speed of the wave front will be described with reference to FIGS. 7A and 7B. FIG. 7A illustrates a case in which a wave front image at a certain time t₁ and a wave front image at a time t₂ (t₁<t₂) are combined into one wave front image 310. Here, it is assumed that there is correspondence information indicating that a wave front 311 at the time t₁ and a wave front 312 at the time t₂ are the same wave front. The propagation analysis unit 15 detects coordinates (xₜ₂, yₜ₂) on the wave front 312 corresponding to coordinates (xₜ₁, yₜ₁) on the wave front 311 from the correspondence information. In this way, it is possible to estimate that a shear wave which has passed through the coordinates (xₜ₁, yₜ₁) at the time t₁ reaches the coordinates (xₜ₂, yₜ₂) at the time t₂. Therefore, the speed v(xₜ₁, yₜ₁) of the shear wave which has passed through the coordinates (xₜ₁, yₜ₁) can be estimated as a value obtained by dividing a distance d between the coordinates (xₜ₁, yₜ₁) and the coordinates (xₜ₂, yₜ₂) by a required time Δt=t₂−t₁. That is, v(xₜ, yₜ)=d/Δt=√{(xₜ₂−xₜ₁)²+(yₜ₂−yₜ₁)²}/Δt is established. The propagation analysis unit 15 performs the above-mentioned process for all of the wave fronts to acquire the speeds of the shear waves for all coordinates through which the wave fronts have passed and stores the speeds of the shear waves.

Returning to FIG. 3, the description will be continued. The first sub-sequence is completed by the above-mentioned operation. Since all of the sub-sequences have not been completed after the first sub-sequence ends (Yes in step S445), a second sub-sequence is performed (step S446).

Then, the second sub-sequence is performed. In the second sub-sequence, the same operation as that in the first sub-sequence except for the characteristics of the push pulse transmitted in step S441 is performed. In the transmission of the push pulse related to the second sub-sequence (i=2 in step S441), the push pulse is transmitted to the center of a small region that is different from that in the first sub-sequence, for example, a focal position 422 illustrated in FIG. 4A. Since the operation in steps S442 to S444 is the same as that in the first sub-sequence, the description thereof will not be repeated.

Hereinafter, similarly, a third sub-sequence and a fourth sub-sequence are performed. In the third sub-sequence, similarly, in the transmission of the push pulse (i=3 in step S441), the push pulse is transmitted to the center of a small region that is different from those in the first sub-sequence and the second sub-sequence, for example, a focal position 432 illustrated in FIG. 4A. In the fourth sub-sequence, similarly, in the transmission of the push pulse (i=4 in step S441), the push pulse is transmitted to the center of a small region that is different from those in the first to third sub-sequences, for example, a focal position 442 illustrated in FIG. 4A.

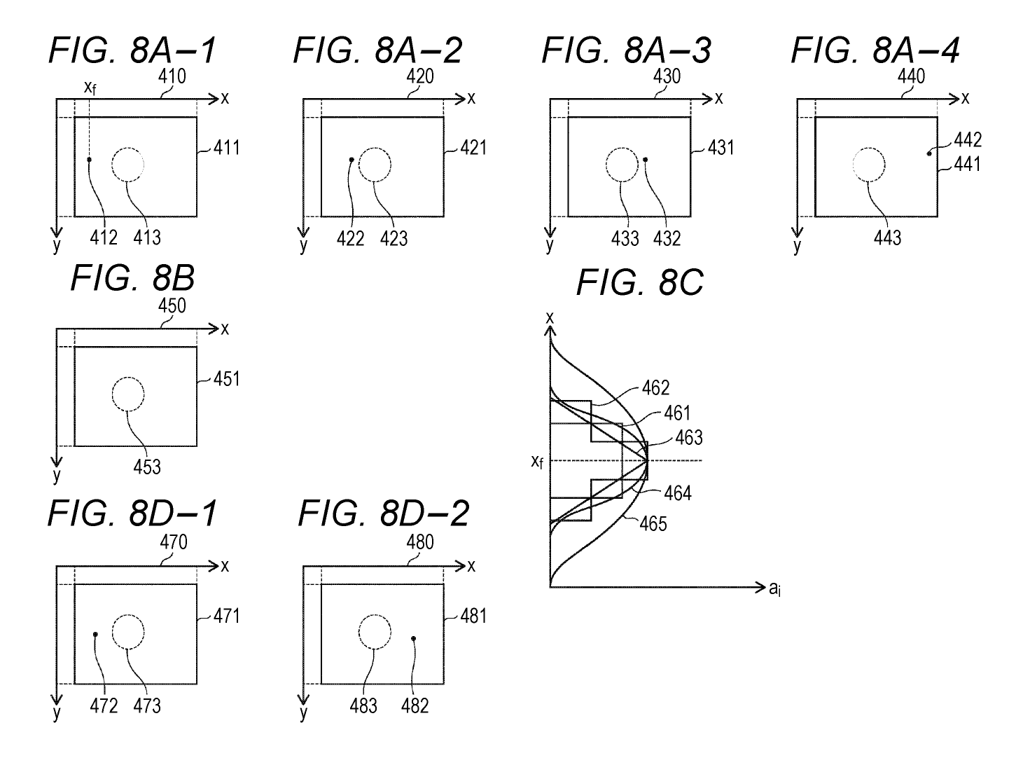

After all of the sub-sequences end (No in step S445), the propagation analysis unit 15 integrates the analysis results of propagation (step S450). Specifically, the propagation analysis unit 15 collects the direction and speed of the shear waves calculated for each sub-sequence and calculates the direction and speed of the shear waves at each coordinate point in the region of interest. This will be described with reference to the schematic diagrams of FIGS. 8A-1 to 8D-2. FIGS. 8A-1 to 8A-4 illustrate the positional relationship among the region of interest, the speed distribution of the shear waves, and the focus of the push pulse in the first to fourth sub-sequences, respectively. For example, in a speed distribution chart 410 illustrated in FIG. 8A-1, a focal position 412 of the push pulse is close to the left end of a region of interest 411 and a region 413 in which the propagation speed of the shear waves is high is detected. Similarly, in a speed distribution chart 420 illustrated in FIG. 8A-2, a focal position 422 of the push pulse is present on the left side of the center of a region of interest 421 and a region 423 in which the propagation speed of the shear waves is high is detected. In addition, in a speed distribution chart 430 illustrated in FIG. 8A-3, a focal position 432 of the push pulse is present on the right side of the center of a region of interest 431 and a region 433 in which the propagation speed of the shear waves is high is detected. Similarly, in a speed distribution chart 440 illustrated in FIG. 8A-4, a focal position 442 of the push pulse is close to the right end of a region of interest 441 and a region 443 in which the propagation speed of the shear waves is high is detected. In practice, the regions 413, 423, 433, and 443 in which the propagation speed of the shear waves is high correspond to one tissue. In some cases, the attenuation of the shear waves causes the boundary between a portion that is close to the focal position of the push pulse and other regions to be clear and causes the boundary between a portion that is far from the focal position of the push pulse and other regions to be unclear. These charts are integrated to create one speed distribution chart 450 illustrated in FIG. 8B. Specifically, the speed of the shear waves at each coordinate point is acquired from the speed distribution charts 410, 420, 430, and 440 and a representative value is calculated. As a method for calculating the representative value, for example, a weighted average or a maximum value may be used, or an average value except for invalid data (for example, since the speed is not capable of being acquired, there is no value and there is a large difference from any of the speeds acquired from other speed distribution charts) may be used. When the weighted average is used, a value that increases as the distance from the focal position of the push pulse decreases and decreases as the distance from the focal position of the push pulse increases can be used as a weighting coefficient aᵢ. The reason is that, as the distance from the focus distance of the push pulse decreases, the energy of the shear waves increases and the accuracy of the speed of the shear waves is expected to be high. As an example of the weighting coefficient aᵢ, 0 may be used as long as the difference between the focal position (the x-coordinate is x_{f}) of the push pulse and the x-coordinate is equal to or greater than a predetermined value as illustrated in a coefficient 461 of FIG. 8C or an arbitrary function that increases as the difference between the focal position (the x-coordinate is x_{f}) of the push pulse and the x-coordinate decreases may be used as illustrated in coefficients 462 to 464 of FIG. 8C. In this case, it is possible to generate the speed distribution chart 450 in which the whole aspect of the region 453 having a high propagation speed of shear waves can be checked.

Finally, the propagation analysis unit 15 generates an elastic image (step S460). Specifically, the propagation analysis unit 15 calculates an elastic modulus for each pixel of the speed distribution chart from the speed of the shear waves and associates each pixel with the elastic modulus to generate an elastic image. An elastic modulus E(xₜ, yₜ) at coordinates (xₜ, yₜ) can be calculated as follows, using the speed v(xₜ, yₜ) of the shear waves at the coordinates:

E(xₜ,yₜ)=2(1+γ)ρ·v(xₜ,yₜ)²

(where γ is the Poisson's ratio of tissues at the coordinates (xₜ, yₜ) and ρ is density).

For example, the elastic modulus may be approximately calculated as follows, using γ=0.5 and ρ=1 g/cm³:

E(xₜ,yₜ)≈3·v(xₜ,yₜ)².

Each pixel and the elastic modulus are associated with each other by, for example, the matching of color information. In this way, for example, as illustrated in FIG. 7B, an elastic image 320 which is color-coded such that a coordinate point where the elastic modulus is equal to or greater than a predetermined value is red, a coordinate point where the elastic modulus is less than the predetermined value is green, and a coordinate point where no elastic modulus is acquired is block is generated. The classification is not limited to binarization and classification and color coding may be performed in predetermined stages. In FIG. 7B, a region 322 in which the elastic modulus is equal to or greater than a predetermined value corresponds to an inclusion 321. In FIG. 7B, the inclusion 321 is illustrated for ease of explanation. However, the inclusion 321 does not directly appear on the actual elastic image. The propagation analysis unit 15 outputs the generated elastic image to the control unit 11 and the control unit 11 outputs the elastic image to the elastic image storage unit 21.

The execution of the operation sequence ends. Then, returning to FIG. 2, the process will be continuously described. Then, the control unit 11 displays the elastic image and the ultrasound image (step S50). Specifically, the control unit 11 performs geometric transformation for the elastic image generated in step S460 and the reference tomographic image signal acquired in step S420 such that the elastic image and the reference tomographic image signal become image data for screen display and outputs the elastic image and the ultrasound image subjected to the geometric transformation to the display unit 3.

The control unit 11 receives information indicating whether to continue the process from the user (operator). When the process is continued, the control unit 11 returns to step S10 and resumes the process. When the process is not continued, the control unit 11 ends the process (step S70).

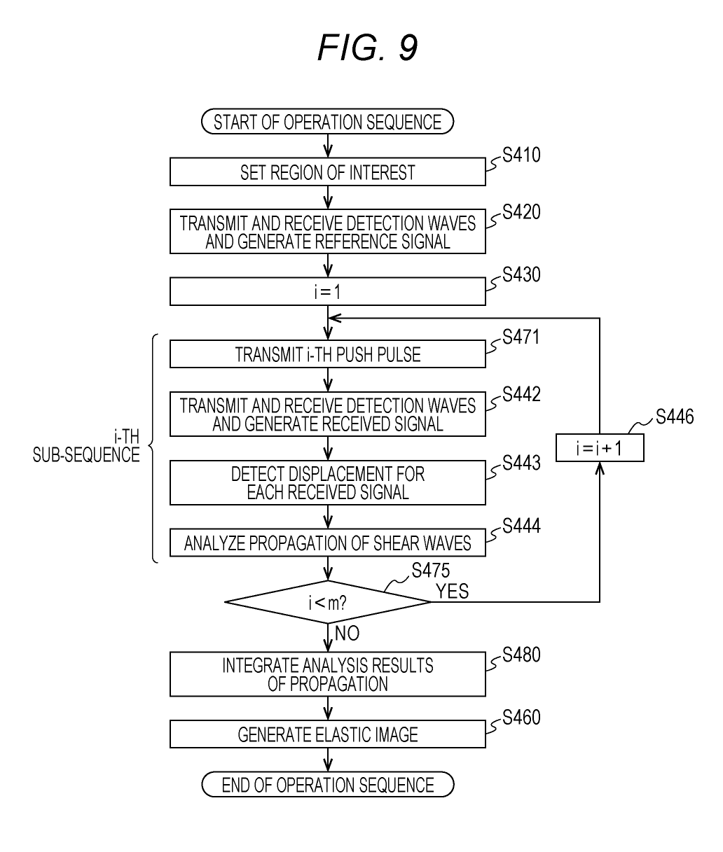

A case in which the second operation sequence (step S60) is performed will be described. FIG. 9 is a flowchart illustrating in detail the second operation sequence. The same operations as those in FIG. 3 are denoted by the same step numbers and the description thereof will not be repeated. The second operation sequence is the same as the first operation sequence except that steps S471, S475, and S480 are performed, instead of steps S441, S445, and S450, and the number of times a sub-sequence including step S471 and steps S442 to S444 is performed is different from that in the first operation sequence. Therefore, hereinafter, the difference between the second operation sequence and the first operation sequence will be described.

In the second operation sequence, since the number of sub-sequences is m (=2) less than n (=4), the focal position of the push pulse is different from that in the first operation sequence. In step S471, for example, the focal position of the first push pulse (i=1) is the center of one of m small regions (here, m is 2) obtained by dividing the region of interest in the column direction of the transducers. A detailed example will be described with reference to FIG. 4B. A region of interest 407 is divided into two small regions 408 and 409 and the first push pulse is transmitted to a focal position 472 in the small region 408. Similarly, the second push pulse (i=2) is transmitted to a focal position 482 in the small region 409.

After all of the sub-sequences end (No in step S475), the propagation analysis unit 15 integrates the analysis results of propagation (step S480). A detailed process is the same as that in step S450 except for the number of sub-sequences. Therefore, as illustrated in FIGS. 8D-1 and 8D-2, there are only two speed distribution charts 470 and 480 which are related to the first sub-sequence and the second sub-sequence, respectively. In the speed distribution chart 470, a focal position 472 of the push pulse is present on the left side of the center of a region of interest 471 and a region 473 in which the propagation speed of the shear waves is high is detected. Similarly, in the speed distribution chart 480, a focal position 482 of the push pulse is present on the right side of the center of a region of interest 481 and a region 483 in which the propagation speed of the shear waves is high is detected. Similarly, in practice, the regions 473 and 483 in which the propagation speed of the shear waves is high correspond to one tissue. These speed distribution charts are integrated to create one speed distribution chart 450 illustrated in FIG. 8B. In this case, when a weighting average is used, the same weighting coefficient as that in step S450 may be used. For example, a coefficient that causes a weighting coefficient to be greater than that in step S450 even though the distance from the focal position of the push pulse is large, specifically, a coefficient 465 illustrated in FIG. 8C may be used. The reason is that the number of targets to be integrated is less than that in step S450 and, when a weighting coefficient for extracting only the vicinity of the focus of the push pulse is used, a region in which the speed is not capable of being acquired is likely to be generated.

As described above, in the second operation sequence, the number of times the sub-sequence is performed is less than that in the first operation sequence. Therefore, it is possible to reduce the time required to generate one elastic image by about m/n times, that is, by ½. As a result, it is possible to improve the frame rate of an elastic image by about n/m times, that is, two times.

<Summary>

According to the above-mentioned structure, when the moving speed of the ultrasound probe is greater than a predetermined threshold value, the operation sequence selection unit selects the second operation sequence in which the number of sub-sequences required to generate one elastic image is small. Therefore, when the moving speed of the ultrasound probe is high, the frame rate of the elastic image can be improved to reduce the influence of the moving speed of the ultrasound probe, such as the deviation between the region of interests in the ultrasound image and the elastic image. In addition, since the frame rate of the elastic image can be improved, it is possible to improve a following performance for the movement of the ultrasound probe. On the other hand, when the moving speed of the ultrasound probe is equal to or less than the predetermined threshold value, the operation sequence selection unit selects the first operation sequence in which the number of sub-sequences required to generate one elastic image is large. When the moving speed of the ultrasound probe is low, it is possible to improve the accuracy of an elastic image. Therefore, the ultrasound diagnostic apparatus according to this embodiment can select an operation sequence that is most suitable for the moving speed of the ultrasound probe and perform the operation sequence.

### Second Embodiment

In the first embodiment, when the moving speed of the ultrasound probe is greater than a predetermined threshold value, the operation sequence selection unit selects the second operation sequence in which the number of sub-sequences required to generate one elastic image is small to improve the frame rate of the elastic image.

In contrast, in this embodiment, a case in which the frame rate of the elastic image is improved by other methods will be described.

<Operation>

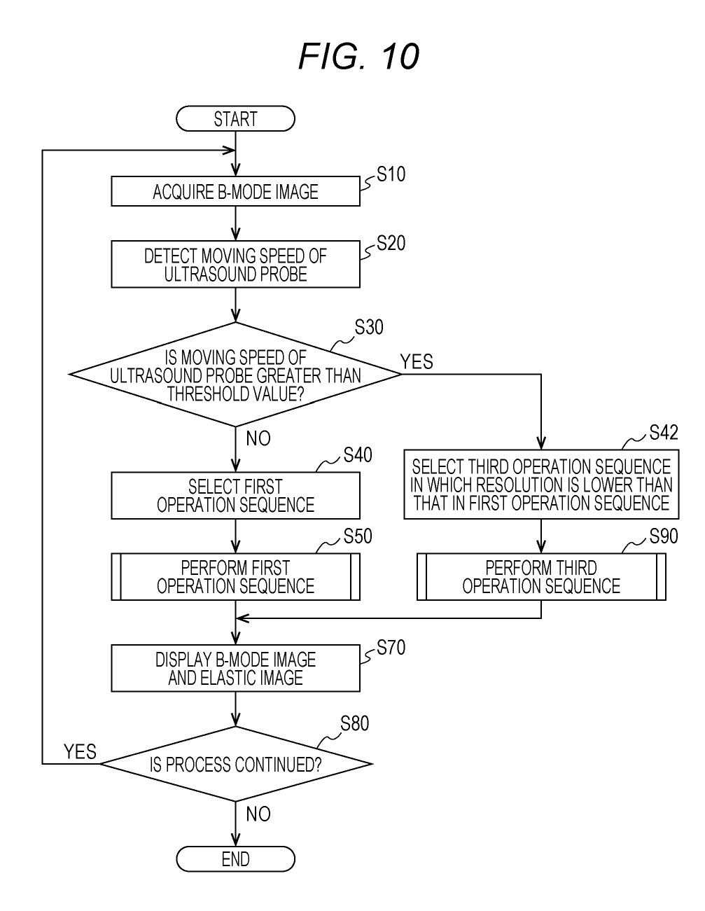

The operation of an ultrasound diagnostic apparatus according to the second embodiment will be described. FIG. 10 is a flowchart illustrating the overall operation of the ultrasound diagnostic apparatus. The same operations as those in FIG. 2 are denoted by the same step numbers and the description thereof will not be repeated.

In the second embodiment, when the moving speed of the ultrasound probe is equal to or less than a predetermined threshold value (No in S30), the exact same operation as that when the moving speed of the ultrasound probe is equal to or less than the predetermined threshold value in the first embodiment is performed. When the moving speed of the ultrasound probe is greater than the predetermined threshold value (Yes in S30), a third operation sequence in which resolution is less than that in the first operation sequence is selected (step S40). Here, the resolution is related to the transmission and reception of detection waves after a push pulse is transmitted, the detection of displacement, and propagation analysis and will be described in detail below.

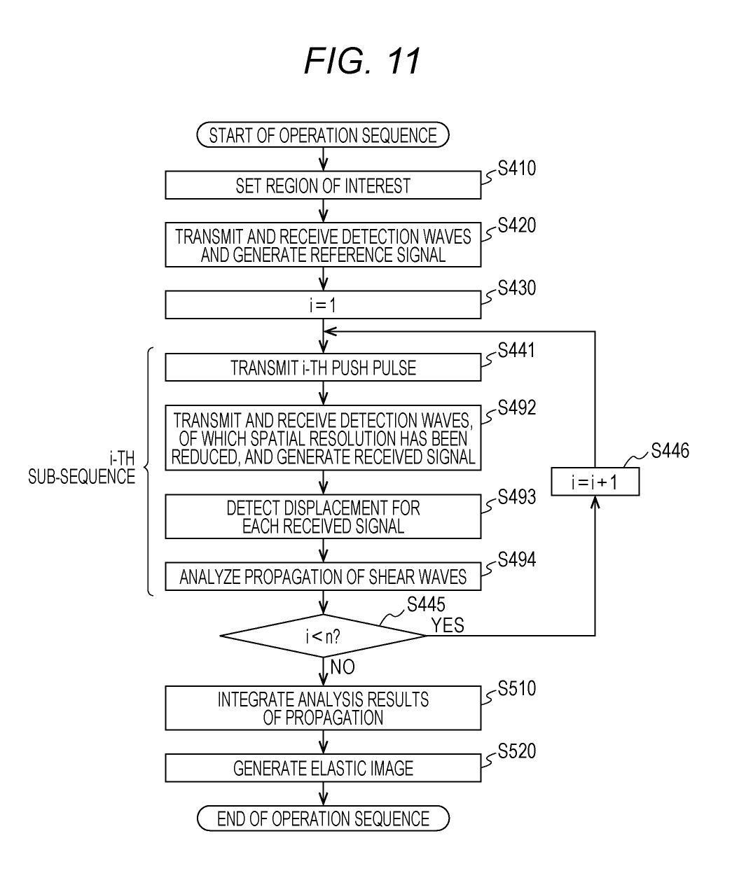

FIG. 11 is a flowchart illustrating in detail the execution (step S90) of the third operation sequence. The same operations as those in FIG. 3 are denoted by the same step numbers and the description thereof will not be repeated. The third operation sequence differs from the first operation sequence only in an operation in steps S492 to S494 related to a sub-sequence, a propagation analysis result integration operation in step S510, and an elastic image generation operation in step S520. Hereinafter, the operation of the third operation sequence will be described. The description of the same operation as that in the first operation sequence will not be repeated and only the difference will be described.

In the sub-sequence of the third operation sequence, the transmission of a push pulse (S441) is the same as that in the first operation sequence and the second operation sequence according to the first embodiment. In contrast, in the transmission and reception of detection waves, the detection of displacement, and shear wave propagation analysis, a process is performed with reduced resolution. Hereinafter, this embodiment will be described in detail below. In this embodiment, a case in which resolution is “spatially” low will be described.

First, ultrasonic waves of which the spatial resolution has been reduced are transmitted to and received from a region of interest a plurality of times and a plurality of acquired ultrasound signals are stored (step S492). Specifically, immediately after the transmission of a push pulse ends, an operation of transmitting and receiving detection waves of which the spatial resolution has been reduced is repeated 10000 times per second. In this way, the tomographic image of a subject is repeatedly acquired until propagation ends immediately after shear waves are generated. An operation related to each transmission event will be described in detail with reference to FIGS. 12A-1 to 12C-2. FIGS. 12A-1 and 12A-2 are schematic diagrams when the transmitted detection waves are plane waves. When the transmitted detection waves are plane waves, in a transmission event related to the first operation sequence, for example, as illustrated in a schematic diagram 510, transmitted detection waves 512 are transmitted by all of the transducer rows of the ultrasound probe or some successive transducers so as to pass through a region of interest 511. In contrast, in a transmission event related to step S492, for example, as illustrated in a schematic diagram 520, every other transducer is selected from all of the transducer rows of the ultrasound probe or some successive transducers and transmitted detection waves 522 are transmitted by only the selected transducers so as to pass through a region of interest 521. In the reception of the reflected detection waves, similarly, in the transmission event related to step S492, every other transducer is selected from all of the transducer rows of the ultrasound probe and an element reception signal is generated by only the selected transducers. An acoustic line signal is generated by reducing resolution in the x direction by half. FIGS. 12B-1 and 12B-2 are schematic diagrams illustrating a case in which the transmitted detection waves are focus waves. When the transmitted detection waves are focus waves, in the transmission event related to the first operation sequence, for example, as illustrated in a schematic diagram 530, a series of focus waves 532 is transmitted so as to scan a region of interest 531. In contrast, in the transmission event related to step S492, for example, as illustrated in a schematic diagram 540, the moving pitch of the focus waves is doubled such that the number of focus waves 532 used for scanning is reduced by half and a region of interest 541 is scanned with the focus waves. In this case, phasing addition is performed whenever a reflected detection wave with respect to one focus wave is received. Therefore, the number of focus waves is reduced by half and the resolution of the acoustic line signal in the x direction is inevitably reduced by half. In this example, every other transducer is selected as the elements for transmitting and receiving plane waves. However, the invention is not limited thereto. For example, every third transducer or every fourth transducer may be selected. In addition, in this embodiment, the moving pitch of the focus waves is doubled. However, the moving pitch may be tripled or quadrupled. In this case, the spatial resolution of the acoustic line signal in the x direction is reduced to ½ to ¼ of the original value. In this way, the amount of calculation required for phasing addition for generating an acoustic line signal is reduced to ½ to ¼ of the original value.

The description will be continued returning to FIG. 11. Then, displacement is detected for each received signal (step S493). A detailed operation is the same as that in step S443. Since the resolution of a tomographic image signal in the x direction is low, the amount of calculation is reduced. As a result, the resolution of an output displacement image in the x direction is also reduced.

Then, shear wave propagation analysis is performed (step S494). A detailed operation is the same as that in step S444. Since the resolution of a displacement image in the x direction is low, the amount of calculation is reduced. As a result, the spatial resolution of the speed of the obtained shear waves is reduced in the x direction.

After all of the sub-sequences end (No in step S445), the propagation analysis unit 15 integrates the analysis results of propagation (step S510). A detailed process is the same as that in step S450. Since the spatial resolution of the speed of the shear waves obtained in each sub-sequence is reduced in the x direction, the amount of calculation is reduced. As a result, the spatial resolution of the speed of the obtained shear waves is also reduced in the x direction.

Finally, the propagation analysis unit generates an elastic image (step S520). A detailed process is the same as that in step S460. Since the spatial resolution of the speed of the shear waves is reduced in the x direction, the amount of calculation is reduced. As a result, the spatial resolution of the obtained elastic image is also reduced in the x direction.

As described above, in the third operation sequence, the amount of calculation required for the sub-sequence is less than that in the first operation sequence and an operation time is reduced. Specifically, the amount of calculation for phasing addition in step S492 and the amount of calculation related to all of steps S493 and S494 and steps 510 to S520 is reduced to ½ to ¼ of the original value. Therefore, it is possible to reduce the time required to generate one elastic image.

### Modification Example

In the second embodiment, in the sub-sequence of the third operation sequence, the “spatial” resolution is reduced in the transmission and reception of detection waves, the detection of displacement, and shear wave propagation analysis. In contrast, in this modification example, in the sub-sequence of the third operation sequence, a case in which “temporal” resolution is reduced in the transmission and reception of detection waves, the detection of displacement, and shear wave propagation analysis will be described.

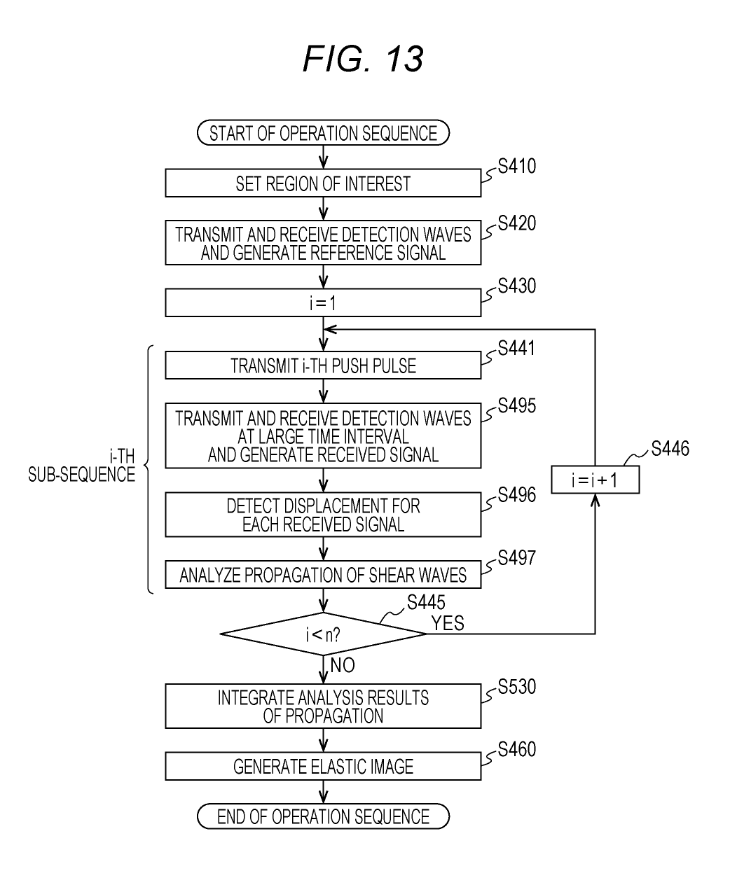

FIG. 13 is a flowchart illustrating in detail the execution of a third operation sequence according to this modification example. The same operations as those in FIGS. 3 and 11 are denoted by the same step numbers and the description thereof will not be repeated. The third operation sequence according to this modification example differs from the third operation sequence according to the second embodiment in that an elastic image is generated in step S460 similarly to the first operation sequence and in an operation in steps S495 to S497 related to a sub-sequence and the integration of the analysis results of propagation in step S530. Since the generation of the elastic image in step S460 has been described in the first embodiment, only the difference between this modification example and the second embodiment will be described below.

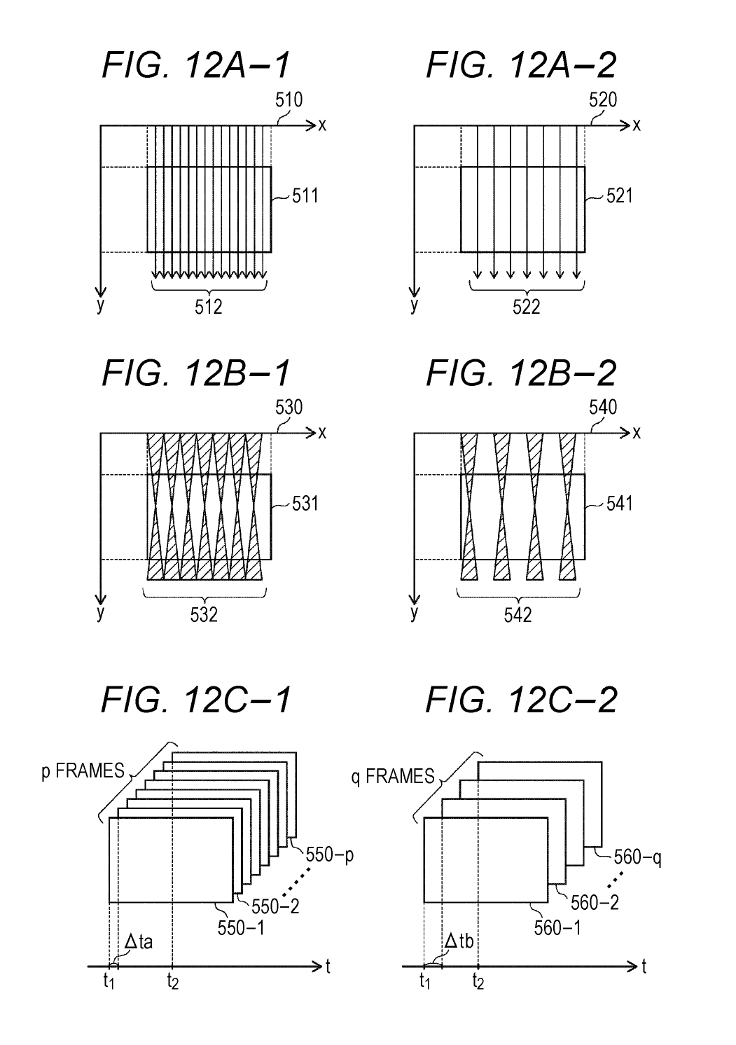

First, ultrasonic waves are transmitted to and received from a region of interest a plurality of times at a large time interval and a plurality of acquired ultrasound signals are stored (step S495). Specifically, immediately after the transmission of a push pulse ends, the transmission and reception of detection waves of which the temporal resolution has been reduced are repeated. The temporal resolution (frame rate) of the detection waves is lower than that in step S442 related to the first operation sequence in order to increase the transmission and reception time interval. This will be described in detail with reference to FIGS. 12C-1 and 12C-2. FIG. 12C-1 is a schematic diagram illustrating step S442 related to the first operation sequence and p ultrasound signals, that is, ultrasound signals 550-1 to 550-p are acquired. An ultrasound signal acquisition time interval Δta is, for example, 100 microseconds (10000 times per second). In contrast, FIG. 12C-2 is a schematic diagram illustrating step S492 related to this modification example and q ultrasound signals, that is, ultrasound signals 560-1 to 560-q are acquired. An ultrasound signal acquisition time interval Δtb is p/q times longer than Δta and is, for example, 200 microseconds (5000 times per second). That is, the time when the acquisition of an ultrasound signal starts and the time when the acquisition of an ultrasound signal ends are constant in step S495 related to this modification example and step S442 related in the first operation sequence and only the time interval at which ultrasound signals are successively acquired, that is, the temporal resolution (frame rate) of the ultrasound signals is different in step S495 and step S442. According to this structure, the number of ultrasound signals to be acquired can be reduced to q/p.

The description will be continued using FIG. 13 again. Then, displacement is detected for each received signal (step S496). A detailed operation is the same as that in step S443. Since the number of received signals is reduced to q/p, the amount of calculation is reduced to q/p and the number of displacement images to be obtained is also reduced to q/p.

Then, shear wave propagation analysis is performed (step S497). A detailed operation is the same as that in step S444. Since the number of displacement images is reduced to q/p, the amount of calculation is reduced to q/p.

As described above, in the operation sequence according to this modification example, the amount of calculation in the sub-sequence is less than that in the first operation sequence and a calculation time is reduced. Specifically, the amount of calculation for phasing addition in step S495 and the amount of calculation related to all of steps S496 and S497 are reduced to q/p. Therefore, it is possible to reduce the time required to generate one elastic image.

<Summary>

According to the above-mentioned structure, when the moving speed of the ultrasound probe is greater than a predetermined threshold value, the operation sequence selection unit selects the third operation sequence in which the (spatial and/or temporal) resolution related to the transmission and reception of detection waves after a push pulse is transmitted, the detection of displacement, and propagation analysis is lower than that in the first operation sequence. Therefore, when the moving speed of the ultrasound probe is high, it is possible to improve the frame rate of an elastic image, similarly to the first embodiment.

### Third Embodiment

In the first embodiment and the second embodiment, when the moving speed of the ultrasound probe is greater than a predetermined threshold value, an operation sequence for improving the frame rate of an elastic image is selected.

In contrast, in this embodiment, a case in which, when the moving speed of the ultrasound probe is greater than a predetermined threshold value, operation sequences using different displacement detection methods are selected will be described.

<Operation>

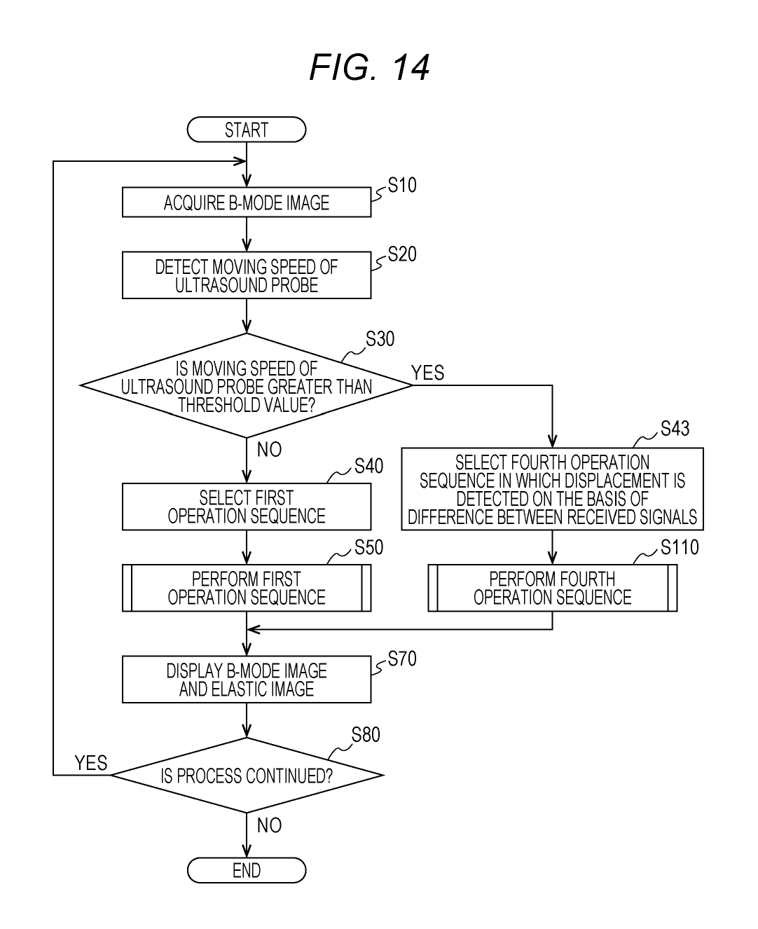

The operation of an ultrasound diagnostic apparatus according to the third embodiment will be described. FIG. 14 is a flowchart illustrating the overall operation of the ultrasound diagnostic apparatus. The same operations as those in FIGS. 2 and 10 are denoted by the same step numbers and the description thereof will not be repeated.

In the third embodiment, when the moving speed of the ultrasound probe is equal to or less than a predetermined threshold value, (No in S30), the exact same operation as that when the moving speed of the ultrasound probe is equal to or less than the predetermined threshold value in the first embodiment is performed. On the other hand, when the moving speed of the ultrasound probe is greater than the predetermined threshold value (Yes in S30), a fourth operation sequence in which displacement is detected on the basis of the difference between the received signals is selected (step S43).

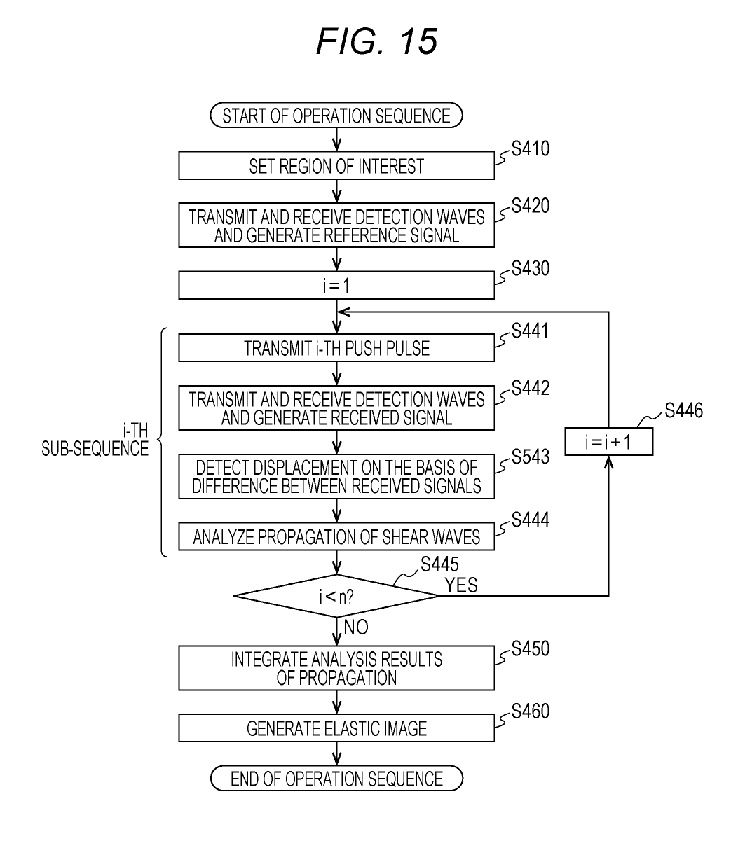

FIG. 15 is a flowchart illustrating in detail the execution (step S110) of the fourth operation sequence. The same operations as those in FIG. 3 are denoted by the same step numbers and the description thereof will not be repeated. The fourth operation sequence differs from the first operation sequence in only an operation in step S543 related to the detection of displacement. Therefore, hereinafter, the difference will be described.

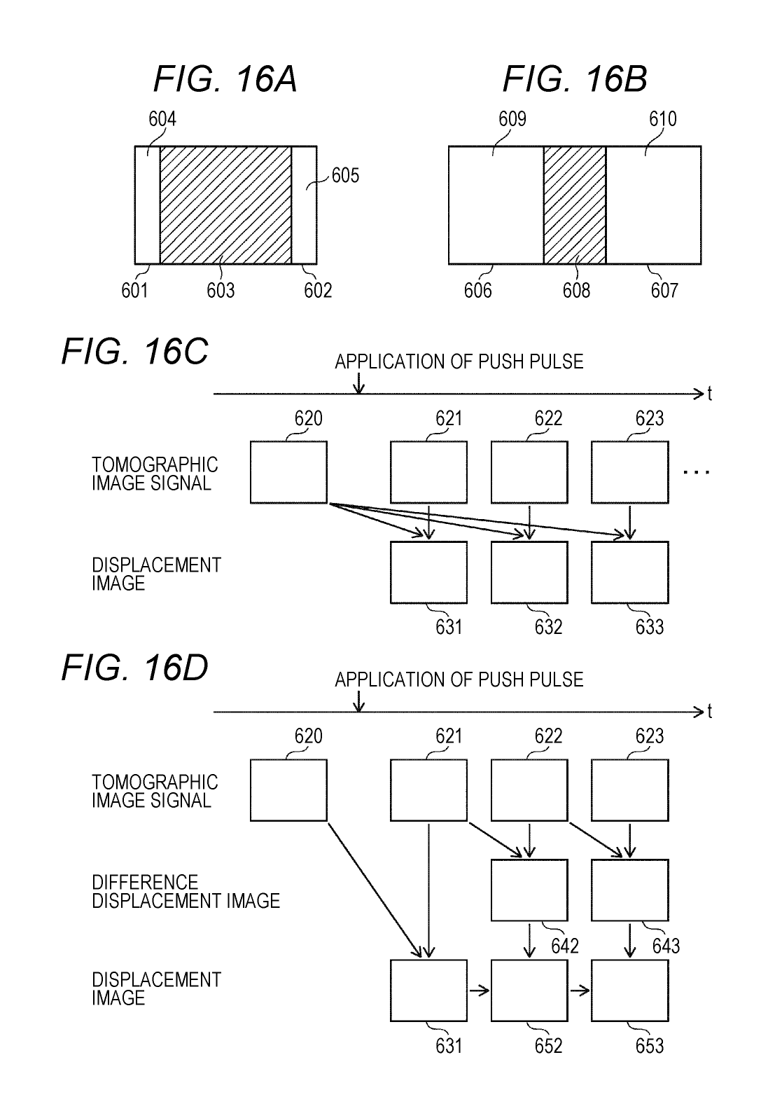

First, before the operation is described, the influence of the moving speed of the ultrasound probe on the detection of displacement will be described. FIGS. 16A and 16B are schematic diagrams when displacement is detected using two ultrasound signals in which the regions of interest are not matched with each other. As described above, displacement is detected by searching for corresponding points between a tomographic image signal, which is a displacement detection target, and a reference tomographic image signal and detecting the difference between the coordinates of the corresponding points. In this case, for example, as illustrated in FIG. 16A, when a region of interest 602 in a tomographic image signal which is a displacement detection target includes a region 603 and a region 605 and a region of interest 601 in a reference tomographic image signal includes the region 603 and a region 604, displacement is detected in the overlap region 603. However, displacement is not capable of being detected in the region 605 since there is no corresponding point in the reference ultrasound signal. As illustrated in FIG. 16B, when a region of interest 607 in a tomographic image signal which is a displacement detection target includes a region 608 and a region 610 and a region of interest 606 in a reference tomographic image signal includes the region 608 and a region 609, the overlap region 608 is narrower than the region 603 and the region 609 in which displacement is not capable of being detected is wider than the region 605. Although not illustrated here, when the region of interest in the tomographic image signal which is a displacement detection target and the region of interest in the reference tomographic image signal does not completely overlap, it is difficult to detect the corresponding points. Therefore, displacement is not detected at all.

Here, the tomographic image signal and the reference tomographic image signal used in the displacement detection method in step S443 related to the first operation sequence are reconfirmed. FIG. 16C is a diagram schematically illustrating a tomographic image signal and a displacement image. As described above, in step S443, the reference tomographic image signal which has been acquired in step S420 performed before the transmission of the push pulse is used. That is, displacement images 631, 632, and 633 are generated, using a reference tomographic image signal 620 acquired in step S420 for tomographic image signals 621, 622, and 623 acquired in step S442, respectively. Therefore, when the ultrasound probe is moved, the overlap area between the regions of interest decreases as the difference between the time when the tomographic image signal is acquired and the time when the reference tomographic image signal is acquired increases. Therefore, the proportion of a region in which displacement is not capable of being detected to the displacement image increases. As a result, it is difficult to perform propagation analysis for a portion through which shear waves pass late, that is, a portion which is far away from the focal position of the push pulse. When the moving speed of the ultrasound probe is high, the reliability of the measurement result of elasticity in a portion that is far away from the focal position of the push pulse is reduced or it is difficult to measure elasticity.

In contrast, a displacement detection method in step S543 related to the fourth operation sequence will be described below. FIG. 16D is a diagram schematically illustrating a tomographic image signal and a displacement image. In step S523, similarly to step S443, the reference tomographic image signal which has been acquired in step S420 performed before the transmission of the push pulse is used as a tomographic image signal which is acquired first. However, for the subsequent tomographic image signal, a tomographic image signal that is one frame before the tomographic image signal is used as the reference tomographic image signal to generate a difference displacement image indicating a relative displacement corresponding to one frame and the difference displacement image is combined with a displacement image corresponding to the tomographic image signal before one frame to generate a displacement image corresponding to the tomographic image signal. Hereinafter, this will be described in detail. First, similarly to step S443, displacement is detected from a tomographic image signal 621 that is acquired first, on the basis of a tomographic image signal 620, and a displacement image 631 is generated. In contrast, displacement is detected from a tomographic image signal 622, on the basis of the tomographic image signal 621 before one frame, and a difference displacement image 642 is generated. Here, displacement indicated by the difference displacement image 642 is the displacement between the tomographic image signal 622 and the tomographic image signal 621. In other words, the displacement is the difference between a displacement image corresponding to the tomographic image signal 622 and a displacement image 631 corresponding to the tomographic image signal 621. Therefore, a composition process which adds the amounts of displacement corresponding to the same pixels in the displacement image 631 and the difference displacement image 642 is performed to acquire a displacement image 652 corresponding to the tomographic image signal 622. Similarly, displacement is detected from a tomographic image signal 623, on the basis of the tomographic image signal 622 before one frame, and a difference displacement image 643 is generated. The displacement image 652 is combined with the difference displacement image 643 to obtain a displacement image 653 corresponding to the tomographic image signal 623. According to this structure, the difference between the times when the reference image signal and the reference tomographic image signal for obtaining a difference displacement image are acquired is always small (the difference is one frame in the above-mentioned example). Therefore, it is possible to maintain the proportion of the overlap area to the region of interest at a high value and to avoid the region in which displacement is not capable of being detected from being extended even if the moving speed of the ultrasound probe is high. When the moving speed of the ultrasound probe is low, this method ideally has the same displacement detection accuracy as the method in step S443. However, in practice, the displacement detection accuracy of this method is generally lower than that in step S443. The reason is as follows. As the difference between the time when the tomographic image signal is acquired and the time when the push pulse is transmitted increases, a larger number of difference displacement images need to be combined. As a result, errors included in the amount of displacement of each difference displacement image are accumulated by addition. Therefore, when the moving speed of the ultrasound probe is low, it is preferable to select the first operation sequence.

### <Summary>

According to the above-mentioned structure, when the moving speed of the ultrasound probe is greater than a predetermined threshold value, the operation sequence selection unit selects the fourth operation sequence in which displacement is detected on the basis of the difference between the received signals. Therefore, when the moving speed of the ultrasound probe is high, it is possible to prevent the generation of a region in which it is difficult to detect displacement.

### Fourth Embodiment

In the first to third embodiments, when the moving speed of the ultrasound probe is greater than a predetermined threshold value, in the operation sequence in which a displacement detection method for improving the frame rate of an elastic image to reduce the influence of the moving speed of the ultrasound probe is selected.

In contrast, in this embodiment, when the moving speed of the ultrasound probe is greater than a predetermined threshold value, an operation sequence which waits for a reduction in the moving speed of the ultrasound probe and then starts to measure elasticity is selected.

<Operation>

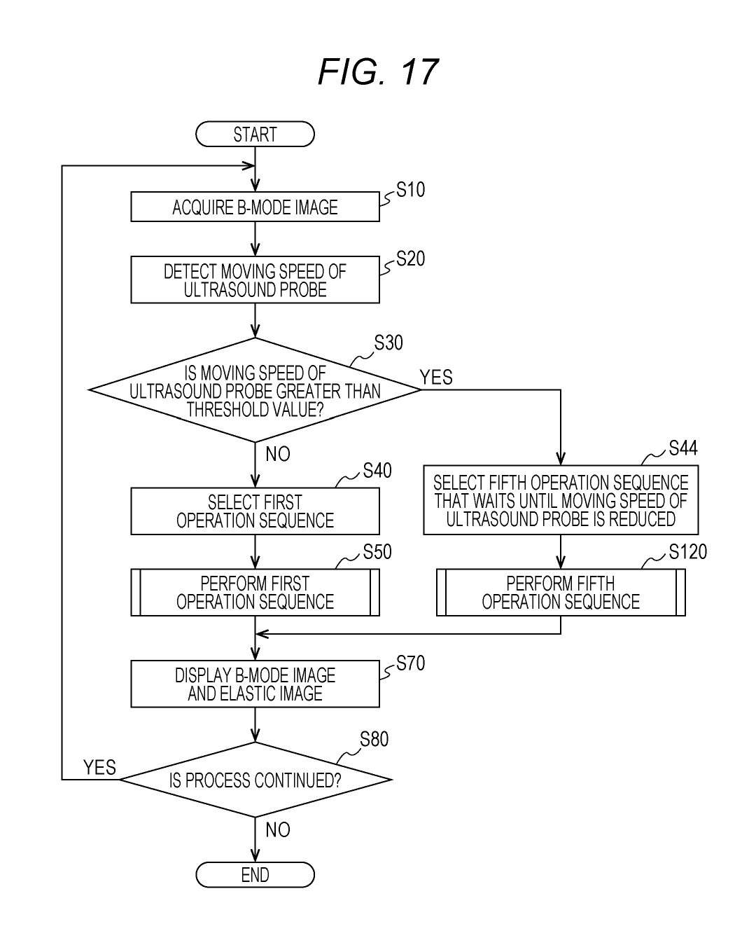

The operation of an ultrasound diagnostic apparatus according to the fourth embodiment will be described. FIG. 17 is a flowchart illustrating the overall operation of the ultrasound diagnostic apparatus. The same operations as those in FIG. 2 are denoted by the same step numbers and the description thereof will not be repeated.

In the fourth embodiment, when the moving speed of the ultrasound probe is equal to or less than a predetermined threshold value, (No in S30), the exact same operation as that when the moving speed of the ultrasound probe is equal to or less than the predetermined threshold value in the first embodiment is performed. On the other hand, when the moving speed of the ultrasound probe is greater than the predetermined threshold value (Yes in S30), a fifth operation sequence which waits until the moving speed of the ultrasound probe is reduced is selected (step S44).

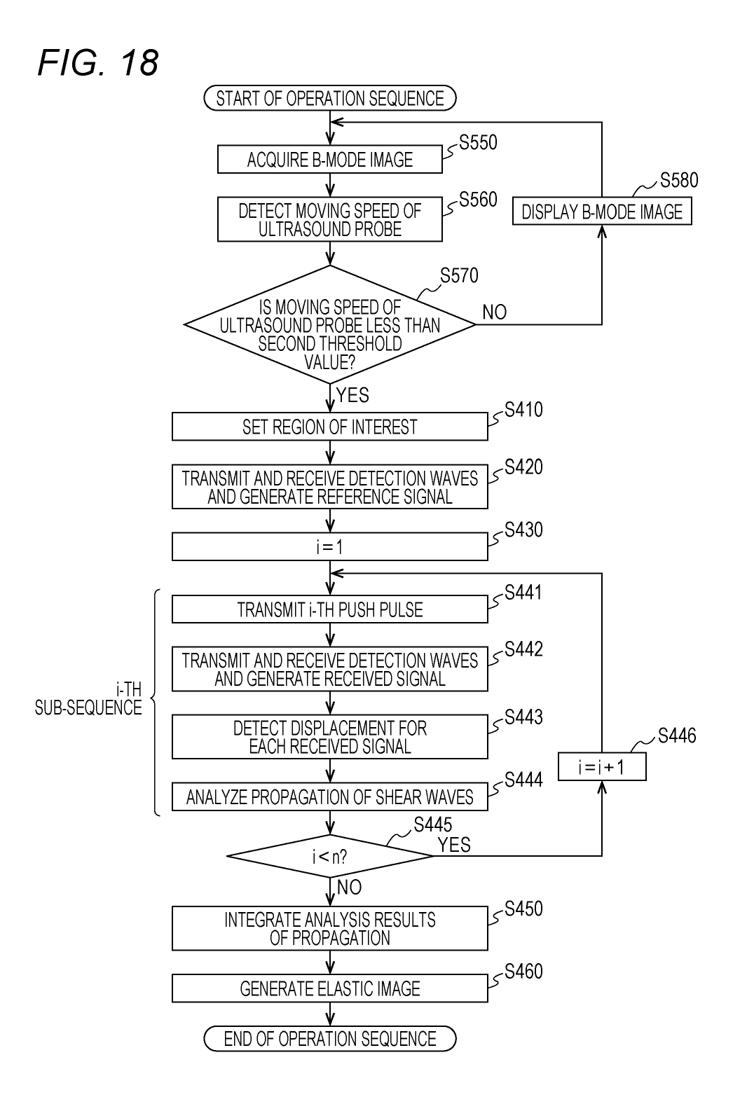

FIG. 18 is a flowchart illustrating in detail the execution (step S120) of the fifth operation sequence. The same operations as those in FIG. 3 are denoted by the same step numbers and the description thereof will not be repeated. The fifth operation sequence differs from the first operation sequence in that steps S550 to S580 are added before a region of interest is set (step S410). Hereinafter, only the difference between the fifth operation sequence and the first operation sequence will be described.

First, ultrasonic waves are transmitted to and received from a subject and an acquired received signal is stored (step S550). Then, the moving speed of the ultrasound probe is detected (step S560). The detailed operations in step S550 and step S560 are the same as those in step S10 and step S20, respectively. Therefore, it is possible to calculate the moving speed of the ultrasound probe.

Then, it is determined whether the moving speed of the ultrasound probe is less than a second threshold value (step S570). The second threshold value is equal to or less than the predetermined threshold value related to step S30. For example, both the predetermined threshold value and the second threshold value are 30 mm/s. Alternatively, for example, the predetermined threshold value may be 30 mm/s and the second threshold value may be 10 mm/s.

When the moving speed of the ultrasound probe is equal to or greater than the second threshold value (No in step S570), only a B-mode image acquired in step S550 is displayed (step S580). The process returns to step S550 and the moving speed of the ultrasound probe is measured again. On the other hand, when the moving speed of the ultrasound probe is less than the second threshold value (Yes in step S570), the process proceeds to step S410 in which the region of interest is set. Therefore, after the moving speed of the ultrasound probe is less than the second threshold value, it is possible to evaluate the hardness of a subject and to prevent propagation analysis for shear waves in a state in which the moving speed of the ultrasound probe is high.

### <Summary>

According to the above-mentioned structure, when the moving speed of the ultrasound probe is greater than a predetermined threshold value, the operation sequence selection unit selects the fifth operation sequence which waits until the moving speed of the ultrasound probe is less than the second threshold value, without starting a sub-sequence. Therefore, when the moving speed of the ultrasound probe is high, only the B-mode image is displayed and a sub-sequence does not start until the moving speed of the ultrasound probe is reduced. As a result, it is possible to prevent propagation analysis for shear waves in a state in which the moving speed of the ultrasound probe is high.

### Other Modification Examples of Embodiments

(1) In the first to fourth embodiments and the modification example, when the moving speed of the ultrasound probe is greater than a predetermined threshold value, anyone of the second to fifth operation sequences is selected. However, combinations of the second to fifth operation sequences may be used. For example, when the moving speed of the ultrasound probe is greater than a predetermined threshold value, an operation sequence in which the number of sub-sequences is small and the spatial resolution of detection waves is low may be used. In this case, when the moving speed of the ultrasound probe is greater than a predetermined threshold value, it is possible to further improve the frame rate of an elastic image. Alternatively, for example, when the moving speed of the ultrasound probe is greater than a predetermined threshold value, an operation sequence in which the temporal resolution of detection waves is low and displacement is detected on the basis of the difference between the received signals may be used. In this case, when the moving speed of the ultrasound probe is greater than a predetermined threshold value, it is possible to compensate the problem that the region in which displacement is not capable of being detected is extended due to a reduction in the temporal resolution of detection waves with a displacement detection method. Alternatively, for example, when the moving speed of the ultrasound probe is greater than a predetermined threshold value (30 mm/s), an operation sequence which waits until the moving speed of the ultrasound probe is less than the second threshold value (10 mm/s), without starting a sub-sequence, and the number of sub-sequences including operations after waiting is small may be used. In this case, when the moving speed of the ultrasound probe is slightly less than the second threshold value, it is possible to control the ultrasound diagnostic apparatus according to the moving speed of the ultrasound probe. Combinations of each embodiment and the modification example are not limited to the above-mentioned examples and any combination of the embodiments and the modification example may be used as long as the obtained effects are not damaged.

The second and third operation sequences according to the first and second embodiments and the modification example each improve the frame rate of the elastic image. However, when only one of the second and third operation sequences is applied or when a combination of two or more of them is applied, among a) a reduction in the number of sub-sequences, b) a reduction in the spatial resolution of the detection waves, c) a reduction in the temporal resolution of the detection waves, b) is more preferable than c) and a) is more preferable than b). For example, when any one of the number of sub-sequences, the spatial resolution of the detection waves, and the temporal resolution of the detection waves is reduced to ¼ of the original value or two or more of them are added and reduced to ¼ of the original value, reducing both the spatial resolution of the detection waves and the temporal resolution of the detection waves to ½ of the original value is preferable to reducing the temporal resolution of the detection waves and it is more preferable to reduce the spatial resolution of the detection waves to ¼ of the original value. In addition, reducing both the spatial resolution of the detection waves and the number of sub-sequences to ½ of the original value is preferable to reducing the spatial resolution of the detection waves to ¼ of the original value and it is more preferable to reduce the number of sub-sequences to ¼ of the original value. The reason is as follows. When the spatial resolution of the detection waves is reduced, the spatial resolution is reduced and the amount of error in the speed of the shear waves increases, which results in a reduction in the accuracy of a hardness evaluation value. In contrast, when the temporal resolution of the detection waves is reduced, the speed of the shear waves is temporally and spatially averaged, which makes it difficult to see hard tissues or the interface with peripheral tissues. Therefore, for the resolution of the detection waves, the deterioration of the quality of an elastic image is more affected by the temporal resolution than the spatial resolution. In addition, even if the number of sub-sequences is reduced, the quality of an elastic image does not deteriorate in a region in which the amplitude of shear waves is sufficiently large. Therefore, unlike a case in which the resolution of the detection waves is reduced, even if the number of sub-sequences is reduced, the quality of an elastic image does not necessarily deteriorate. In addition, even if the resolution of the detection waves is reduced to ¼ of the original value, it is difficult to reduce the number of sub-sequences to ¼ of the original value since only the calculation time is reduced among the times related to one operation sequence. In contrast, when the number of sub-sequences is reduced to ¼ of the original value, the time required for a sub-sequence that makes up most of the time related to one operation sequence is reduced to ¼ of the original value. Therefore, the frame rate can be substantially quadrupled.

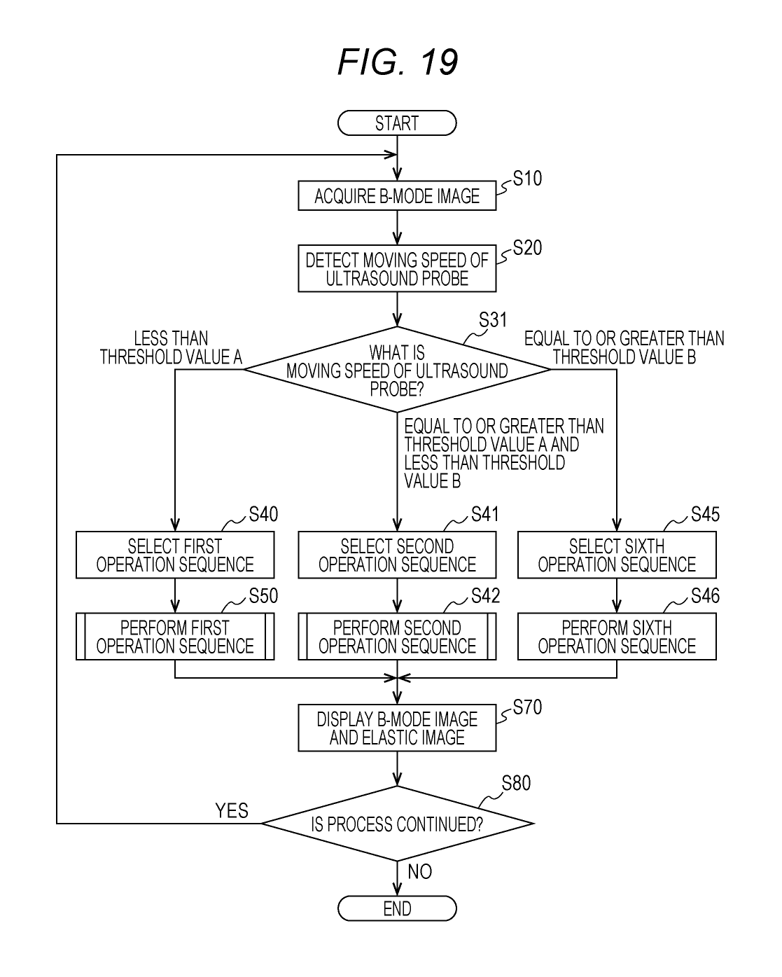

(2) In the first to fourth embodiments and the modification example, only one of two types of operation sequences is selected using only one predetermined threshold value. However, an operation sequence that is most suitable for the moving speed of the ultrasound probe may be selected using a plurality of threshold values. For example, as illustrated in FIG. 19, a threshold value A and a threshold value B that is greater than the threshold value A may be used. In this case, when the moving speed of the ultrasound probe is less than the threshold value A in step S31, the first operation sequence is selected. When the moving speed of the ultrasound probe is equal to or greater than the threshold value A and is less than the threshold value B, the second operation sequence is selected. When the moving speed of the ultrasound probe is equal to or greater than the threshold value B, the sixth operation sequence is selected. In this case, the threshold value A and the threshold value B are, for example, 10 mm/s and 20 mm/s, respectively. The sixth operation sequence is a combination of the second operation sequence and the third operation sequence, that is, an operation sequence in which both the number of sub-sequences and the spatial resolution of detection waves are reduced. In this way, it is possible to select an operation sequence that is most suitable for the moving speed of the ultrasound probe.

In the fifth operation sequence, similarly, a plurality of second threshold values may be used. For example, when a predetermined threshold value for selecting the fifth operation sequence is 30 mm/s, two second threshold values of 20 mm/s and 10 mm/s are used. When the moving speed of the ultrasound probe is equal to or less than 10 mm/s, the same process as that in the first operation sequence is continued. When the moving speed of the ultrasound probe is greater than 10 mm/s and is equal to or less than 20 mm/s, the same process as that in the second operation sequence is continued. In this way, it is possible to select an operation sequence that is most suitable for the moving speed of the ultrasound probe.

(3) In each embodiment and the modification example, shear wave propagation analysis is performed in the order of the displacement region extraction process, the thinning process, the spatial filtering process, and the temporal filtering process. However, the shear wave propagation analysis may be performed in the order of a process of detecting the peak time of the displacement of each portion, the temporal filtering process, and the spatial filtering process.

(4) In each embodiment and the modification example, when the moving speed of the ultrasound probe is equal to or less than a predetermined threshold value, the first operation sequence is used. However, for example, an operation sequence in which the moving speed of the ultrasound probe is low and the accuracy of measurement is improved may be used. As an operation sequence for improving the accuracy of measurement, for example, a synthetic aperture method can be used in the transmission and reception of detection waves. In this way, when the moving speed of the ultrasound probe is low, it is possible to improve the accuracy of evaluating the hardness of tissues.

(5) In each embodiment and the modification example, the displacement image or the difference displacement image is generated on the basis of the difference between the tomographic image signal and the reference tomographic image signal. However, the invention is limited to this case. When the displacement image or the difference displacement image is generated, a correction process that excludes the moving speed of the ultrasound probe 2 from displacement may be performed. This correction process may be based on displacement at a position that is deeper or shallower than the focal position of a push pulse as disclosed in, for example, JP 2013-544615 A, or may be the same as the process of detecting the moving speed of the ultrasound probe 2 in step S20. For example, when the moving speed of the ultrasound probe 2 is detected on the basis of the measured value of a sensor provided in the ultrasound probe 2, displacement may be corrected on the basis of the measured value of the sensor.

(6) In each embodiment and the modification example, when the moving speed of the ultrasound probe 2 is detected on the basis of the tomographic image signal, the probe movement detection unit 16 detects the moving speed of the ultrasound probe 2 from the difference between the latest tomographic image signal and the previous tomographic image signal. However, the invention is not limited to this case. For example, the probe movement detection unit 16 may detect the moving speed of the ultrasound probe 2 from the difference between the latest tomographic image signal and a tomographic image signal that is two or more frames before the latest tomographic image signal. Alternatively, for example, the probe movement detection unit 16 may direct the displacement detection unit 14 to detect the displacement of the latest tomographic image signal, using a tomographic image signal that is one frame (or two or more frames) before the latest tomographic image signal as the reference tomographic image signal and may detect the moving speed of the ultrasound probe 2 on the basis of a displacement image.

(7) In the first embodiment, the number of sub-sequences is 4 in the first operation sequence and is 2 in the second operation sequence. However, the invention is not limited to this case. The first operation sequence and the second operation sequence may have any number of sub-sequences as long as the number of sub-sequences in the second operation sequence is less than that in the first operation sequence. For example, the number of sub-sequences is 5 in the first operation sequence and is 3 in the second operation sequence. The number of sub-sequences in the second operation sequence may be 1. In this case, in the second operation sequence, steps S430, S445, and S446 related to a loop process are not necessary. In addition, since it is not necessary to integrate the analysis results of propagation, the second operation sequence may not include step S450.

(8) In the first embodiment, the focal positions of the push pulses are set in different portions in the first operation sequence and the second operation sequence. However, the invention is not limited to this case. For example, in the second operation sequence, a necessary number of positions may be selected from the focal positions of the push pulses in the first operation sequence and the selected positions may be used as the focal positions of the push pulses.

In all of the operation sequences, the focal position of the push pulse is not limited to the center of the small region. For example, any position of the small region may be used as the focal position of the push pulse. Alternatively, for example, the small regions may be set such that an overlap area is generated and any position of the small region may be used as the focal position of the push pulse.

(9) In the second embodiment, when the detection waves of which the spatial resolution has been reduced are transmitted and received to generate a received signal, the transducers of the ultrasound probe 2 used to transmit and receive the detection waves are thinned out. However, other methods may be used to thin out the transducers. For example, the same process as that in step S442 related to the first operation sequence may be performed to transmit the detection waves and the transducers may be thinned out for only the reception of the reflected detection waves. Alternatively, for example, when the transducers of the ultrasound probe 2 used to transmit and receive the detection waves are not thinned out and phasing addition is performed to generate an acoustic line signal, every second to fourth acoustic line signal may be generated in the direction (x direction) of the transducer column in step S442 related to the first operation sequence to thin out the acoustic line signals per tomographic image signal to ½ to ¼ of the original value.

Alternatively, when phasing addition is performed to generate an acoustic line signal, the transducers may be thinned out in the depth direction (y direction). In this case, it is possible to further reduce the amount of calculation.

(10) In the fourth operation sequence according to the third embodiment, a difference displacement image corresponding to a tomographic image signal is generated on the basis of the previous tomographic image signal. However, the invention is not limited to this case. For example, the difference displacement image may be generated on the basis of any tomographic image signal acquired in step S442. In this case, it is possible to reduce the number of times the difference displacement image is combined and to reduce a displacement error. Specifically, for example, for a tomographic image signal in a twenty-second frame acquired in step S442, three difference displacement images, that is, a difference displacement image corresponding to a tomographic image signal in a first frame and a tomographic image signal in an eleventh frame, a difference displacement image corresponding to the tomographic image signal in the eleventh frame and a tomographic image signal in a twenty-first frame, and a difference displacement image corresponding to the tomographic image signal in the twenty-first frame and the tomographic image signal in the twenty-second frame are combined with a displacement image corresponding to the tomographic image signal in the first frame. In this way, the number of times the difference displacement image is combined can be reduced to 3.

(11) In each embodiment and the modification example, an operation sequence is selected on the basis of whether the moving speed of the ultrasound probe is greater than a predetermined threshold value. However, the invention is not limited to this case. For example, an operation sequence may be selected on the basis of whether the moving speed of the ultrasound probe is greater than a predetermined threshold value for a predetermined period of time or more. Here, the predetermined period of time is, for example, 3 seconds. Alternatively, for example, for the moving speed of the ultrasound probe, an operation sequence may be selected on the basis of whether both the latest instantaneous speed and an average speed for the late 3 seconds are greater than a predetermined threshold value.

Similarly, in the fifth operation sequence according to the fourth embodiment, for example, when the moving speed of the ultrasound probe is less than the second threshold value for a predetermined period of time or more, the sub-sequence may start. Alternatively, for example, for the moving speed of the ultrasound probe, when both the latest instantaneous speed and the average speed for the late 3 seconds are less than the second threshold value, the sub-sequence may start.

In this case, it is possible to avoid a situation in which, when the moving speed of the ultrasound probe is temporarily reduced while the operator is moving the ultrasound probe, the ultrasound diagnostic apparatus erroneously detects that the moving speed of the ultrasound probe is low and selects an operation sequence that is not suitable for the moving speed of the ultrasound probe or starts a sub-sequence.

(12) In each embodiment and each modification example, the ultrasound diagnostic apparatus generates an elastic image for each operation sequence and displays the elastic image. However, the invention is not necessarily limited to this case. For example, the ultrasound diagnostic apparatus may perform the process of creating the elastic and the process of storing the elastic image in the elastic image storage unit and may not perform the process of displaying the elastic image. In addition, the ultrasound diagnostic apparatus may output the elastic image to, for example, an external display device or an external image processing device. Alternatively, for example, the ultrasound diagnostic apparatus may perform only the process of analyzing the propagation state of the shear waves for each operation sequence and may store the analysis result of the propagation state of the shear waves, such as a speed distribution chart indicating the speed of the shear waves, in the elastic image storage unit. In this case, the ultrasound diagnostic apparatus may generate an elastic image from the analysis result of the propagation state of the shear waves, if necessary. Alternatively, the ultrasound diagnostic apparatus may output the analysis result of the propagation state of the shear waves to another apparatus and the apparatus may generate or display an elastic image.

In each embodiment and each modification example, after one operation sequence ends, the ultrasound diagnostic apparatus performs the next operation sequence. However, the invention is not necessarily limited to this case. For example, the ultrasound diagnostic apparatus may perform the operation sequence a predetermined number of times or the number of times designated by the user.

(13) In each embodiment and each modification example, the ultrasound diagnostic apparatus is connected to the display unit 3. However, the invention is not necessarily limited to this case. For example, the ultrasound diagnostic apparatus 1 may include the display unit 3. Alternatively, the ultrasound diagnostic apparatus 1 may not be connected to the display unit 3 and may store the elastic image, which has been generated by the propagation analysis unit 15 and then stored in the elastic image storage unit 21, in another storage medium or output the elastic image to another apparatus through a network.

Similarly, the ultrasound diagnostic apparatus may include the ultrasound probe 2. Alternatively, the ultrasound probe 2 may include the ultrasound signal acquisition unit 13 and an ultrasound diagnostic apparatus without the ultrasound signal acquisition unit 13 may acquire an acoustic line signal from the ultrasound probe 2.

(14) In the ultrasound diagnostic apparatuses according to each embodiment and each modification example, all or some of the components may be implemented by an integrated circuit including one chip or a plurality of chips, a computer program, or other structures. For example, the propagation analysis unit and an evaluation unit may be implemented by one chip. In addition, only the ultrasound signal acquisition unit may be implemented by one chip and the displacement detection unit may be implemented by another chip.

When the components are implemented by an integrated circuit, typically, a large scale integration (LSI) circuit is used as the integrated circuit. Here, the LSI circuit is used as the integrated circuit. The LSI circuits are classified into an IC, a system. LSI circuit, a super LSI circuit, and an ultra LSI circuit according to the degree of integration.

An integration method is not limited to LSI and may be achieved by a dedicated circuit or a general-purpose processor. After the LSI circuit is manufactured, a field programmable gate array (FPGA) that is programmable or a reconfigurable process in which the connection or setting of circuit cells in the LSI circuit is reconfigurable may be used.

When an integration technique that replaces LSI appears with the progress of a semiconductor technique or other techniques derived from the semiconductor technique, the functional blocks may be integrated by the integration technique.

In addition, the ultrasound diagnostic apparatuses according to each embodiment and each modification example may be implemented by a program that is written to a storage medium or a computer that reads and executes the program. The storage medium may be any recording medium such as a memory card or a CD-ROM. The ultrasound diagnostic apparatus according to the embodiment of the invention may be implemented by a program that is downloaded through a network and a computer that downloads the program from the network and executes the program.

(15) All of the above-described embodiments indicate preferred embodiments of the invention. For example, the numerical values, the shapes, the materials, the components, the arrangement position and connection form of the components, the processes, and the order of the processes described in the embodiments are illustrative and do not limit the scope and spirit of the invention. In addition, among the components of the embodiments, the processes which are not described in an independent claim indicating the most generic concept of the invention described as arbitrary components forming preferred embodiments.

In some cases, the scales of the components described in the drawings in each embodiment are different from the actual scales for ease of understanding of the invention. In addition, the invention is not limited by each of the above-described embodiments and can be appropriately changed without departing from the scope and spirit of the invention.

In addition, in the ultrasound diagnostic apparatus, members, such as circuit parts and lead lines, are present on a substrate. Electrical wires and electric circuits can be embodied in various ways on the basis of general knowledge in this technical field and are not directly connected with the invention. Therefore, the description thereof will be omitted. The above-mentioned diagrams are schematic diagrams in which components are schematically illustrated.

### APPENDIX

(1) According to an embodiment, there is provided an ultrasound diagnostic apparatus that transmits a push pulse for focusing ultrasonic waves on a specific part of a subject to physically press tissues in the specific part using an ultrasound probe, repeatedly transmits and receives the ultrasonic waves to and from the subject using the ultrasound probe, and detects a propagation state of shear waves generated from the pressed tissues of the specific part, which are a vibration source, in a region of interest set in the subject. The ultrasound diagnostic apparatus includes: a push pulse transmission unit that transmits the push pulse; a displacement detection unit that transmits detection waves to the subject a plurality of times after the push pulse is transmitted, receives reflected detection waves corresponding to the detection waves from the subject, generates a plurality of received signals in time series, and detects displacement of the tissues in the subject due to the shear waves caused by the push pulse at each time when the reflected detection waves are received; an elasticity measurement unit that analyzes the propagation state of the shear waves in the region of interest on the basis of a detection result of the displacement detection unit and measures elasticity of each tissue in the subject; a probe movement detection unit that detects a moving speed of the ultrasound probe; a sequence holding unit that holds a plurality of operation sequences defining a series of operations performed by the push pulse transmission unit, the displacement detection unit, and the elasticity measurement unit in cooperation with each other which enables the elasticity measurement unit to measure the elasticity; and a sequence selection unit that selects one operation sequence from the plurality of operation sequences held by the sequence holding unit on the basis of a detection result of the probe movement detection unit.

According to another embodiment, there is provided an ultrasound signal processing method that transmits a push pulse for focusing ultrasonic waves on a specific part of a subject to physically press tissues in the specific part using an ultrasound probe, repeatedly transmits and receives the ultrasonic waves to and from the subject using the ultrasound probe, and detects a propagation state of shear waves generated from the pressed tissues of the specific part, which are a vibration source, in a region of interest set in the subject. The ultrasound signal processing method includes: detecting a moving speed of the ultrasound probe; selecting an operation sequence defining a series of operations for measuring elasticity in the region of interest in the subject from a plurality of operation sequences which are held in advance, on the basis of the moving speed of the ultrasound probe; and transmitting the push pulse, transmitting detection waves to the subject a plurality of times after the push pulse is transmitted, receiving reflected detection waves corresponding to the detection waves from the subject, generating a plurality of received signals in time series, detecting displacement of the tissues in the subject due to the shear waves caused by the push pulse at each time when the reflected detection waves are received, and analyzing the propagation state of the shear waves in the region of interest on the basis of the displacement of the tissues in the subject to measure elasticity of each tissue in the subject, according to the selected operation sequence.

According to the present disclosure, the above-mentioned structure makes it possible to change the operation sequence for evaluating hardness on the basis of the moving speed of the ultrasound probe. Therefore, when the moving speed of the ultrasound probe is high, for example, it is possible to select an operation sequence that is not affected by the movement of the ultrasound probe. On the other hand, when the moving speed of the ultrasound probe is low, for example, it is possible to select an operation sequence that is affected by the movement of the ultrasound probe, but improves the accuracy of measurement. Therefore, the operator can move the ultrasound probe, without considering the operation state of the ultrasound diagnostic apparatus, and convenience is improved.

(2) In the ultrasound diagnostic apparatus according to (1), when the moving speed of the ultrasound probe is greater than a predetermined speed, the sequence selection unit may select an operation sequence such that the influence of the moving speed of the ultrasound probe on the measurement result of the elasticity measurement unit is small.

According to this structure, when the ultrasound diagnostic apparatus detects the propagation state of the shear waves, regardless of the moving speed of the ultrasound probe, it is possible to prevent the influence of the moving speed of the ultrasound probe on the detection of the propagation state.

(3) The ultrasound diagnostic apparatus according to (2) may further include an elastic image generation unit that generates one elastic image indicating the elasticity of each tissue in the subject for one operation sequence, on the basis of the measurement result of the elasticity measurement unit. When the moving speed of the ultrasound probe is greater than the predetermined speed, the sequence selection unit may select an operation sequence such that a frame rate of the elastic image is improved.

According to this structure, when the moving speed of the ultrasound probe is greater than the predetermined speed, it is possible to improve a following performance for the movement of the ultrasound probe.

(4) In the ultrasound diagnostic apparatus according to (2) and (3), the operation sequence may include one push pulse transmission operation, an operation of detecting displacement corresponding to the push pulse, and an operation of measuring elasticity based on the detected displacement. When the moving speed of the ultrasound probe is greater than the predetermined speed, the sequence selection unit may select an operation sequence such that at least one of a spatial throughput and a temporal throughput is reduced in the detection of the displacement by the displacement detection unit and the time required for one operation sequence is reduced.

According to this structure, it is possible to reduce the calculation time in the detection of displacement and shear wave propagation analysis. Therefore, it is possible to improve the frame rate of the elastic image and to prevent the influence of the moving speed of the ultrasound probe on the detection of the propagation state of the shear waves.

(5) In the ultrasound diagnostic apparatus according to (2) and (3), the operation sequence may include two or more sub-sequences. Each sub-sequence may include one push pulse transmission operation, an operation of detecting displacement corresponding to the push pulse, and an operation of measuring elasticity based on the detected displacement. A position on which the push pulse is focused may vary depending on the sub-sequence. The operation sequence may further include a process of measuring the elasticity of each tissue in the subject on the basis of the elasticity measured for each sub-sequence. The sequence selection unit may select an operation sequence such that at least one of (a) when the moving speed of the ultrasound probe is greater than the predetermined speed, at least one of a spatial throughput and a temporal throughput is reduced in the detection of the displacement by the displacement detection unit and the time required for one sub-sequence is reduced and (b) the number of sub-sequences included in one operation sequence is reduced is satisfied.

According to this structure, the time required for one operation sequence is shortened by reducing the calculation time in the detection of displacement and shear wave propagation analysis and/or by reducing the number of sub-sequences. Therefore, it is possible to improve the frame rate of the elastic image and to prevent the influence of the moving speed of the ultrasound probe on the detection of the propagation state of the shear waves.

(6) In the ultrasound diagnostic apparatus according to any one of (2) to (5), the operation sequence may be defined by parameters for designating a temporal resolution indicating how frequently the displacement detection unit generates a received signal and detects displacement and a spatial resolution when the displacement detection unit detects the displacement. When the moving speed of the ultrasound probe is greater than the predetermined speed, the sequence selection unit may select an operation sequence such that at least one of the temporal resolution and the spatial resolution is reduced.

According to this structure, since the detection of displacement and shear wave propagation analysis are temporally or spatially performed, it is possible to reduce the amount of calculation. Therefore, the calculation time is reduced in the detection of displacement and shear wave propagation analysis and it is possible to improve the frame rate of the elastic image. It is possible to prevent the influence of the moving speed of the ultrasound probe on the detection of the propagation state of the shear waves.

(7) In the ultrasound diagnostic apparatus according to any one of (2) to (6), when the moving speed of the ultrasound probe is equal to or less than the predetermined speed, the sequence selection unit may select an operation sequence such that a spatial resolution of the received signal generated by the displacement detection unit is improved.

According to this structure, when the moving speed of the ultrasound probe is equal to or less than the predetermined speed, it is possible to detect the propagation state of the shear waves with high accuracy.

(8) In the ultrasound diagnostic apparatus according to any one of (2) to (7), in a case in which the sequence selection unit is set so as to select a first operation sequence when the moving speed of the ultrasound probe is greater than a first speed and to select a second operation sequence when the moving speed of the ultrasound probe is equal to or less than the first speed, when the moving speed of the ultrasound probe is greater than a second speed greater than the first speed, the sequence selection unit may select a third operation sequence, instead of the first operation sequence.

According to this structure, it is possible to select an operation sequence that is most suitable for the moving speed of the ultrasound probe.

(9) In the ultrasound diagnostic apparatus according to (1), when the moving speed of the ultrasound probe is greater than a first speed, the sequence selection unit may select an operation sequence which does not start until the moving speed of the ultrasound probe is equal to or less than a second speed.

According to this structure, when the moving speed of the ultrasound probe is high, the ultrasound diagnostic apparatus waits until the moving speed of the ultrasound probe is reduced. Therefore, when the ultrasound diagnostic apparatus detects the propagation state of the shear waves, it is possible to prevent the influence of the moving speed of the ultrasound probe on the detection of the propagation state.

(10) In the ultrasound diagnostic apparatus according to (9), the second speed may be equal to or less than the first speed.

According to this structure, ultrasound diagnostic apparatus can detect the propagation state of the shear waves after the moving speed of the ultrasound probe is reduced until the influence of the moving speed of the ultrasound probe is removed.

(11) In the ultrasound diagnostic apparatus according to (1), the sequence selection unit may select an operation sequence such that the displacement detection unit generates a reference signal before the push pulse is transmitted and detects displacement using a difference between the reference signal and the received signal when the moving speed of the ultrasound probe is equal to or less than a predetermined speed; and the displacement detection unit detects displacement, using a difference between a plurality of received signals which are arranged in time series as an amount of change in displacement, when the moving speed of the ultrasound probe is greater than the predetermined speed.

According to this structure, when the moving speed of the ultrasound probe is high, displacement detection can be performed such that an area in which displacement can be detected is not reduced.

(12) In the ultrasound diagnostic apparatus according to any one of (1) to (11), the probe movement detection unit may detect the moving speed of the ultrasound probe on the basis of the received signal acquired by the ultrasound probe.

According to this structure, it is possible to detect the moving speed of the ultrasound probe, without providing, for example, a sensor in the ultrasound probe.

(13) In the ultrasound diagnostic apparatus according to any one of (1) to (11), the probe movement detection unit may detect the moving speed of the ultrasound probe on the basis of a signal acquired from a sensor that is provided inside or outside the ultrasound probe.

According to this structure, the ultrasound diagnostic apparatus does not need to perform an operation for calculating the moving speed of the ultrasound probe and can accurately detect the moving speed of the ultrasound probe.

The ultrasound diagnostic apparatus and the ultrasound signal processing method according to the embodiments of the present disclosure are useful to measure the hardness of tissues using ultrasonic waves. Therefore, it is possible to improve the accuracy of measuring the hardness of tissues and the ultrasound diagnostic apparatus and the ultrasound signal processing method can be effectively used in, for example, medical diagnostic apparatuses.

Although the present invention has been described and illustrated in detail, it is clearly understood that the same is by way of illustrated and example only and is not to be taken by way of limitation, the scope of the present invention being interpreted by terms of the appended claims.

What is claimed is:

1. An ultrasound diagnostic apparatus that transmits a push pulse for focusing ultrasonic waves on a specific part of a subject to physically press tissues in the specific part using an ultrasound probe, repeatedly transmits and receives the ultrasonic waves to and from the subject using the ultrasound probe, and detects a propagation state of shear waves generated from the pressed tissues of the specific part, which are a vibration source, in a region of interest set in the subject, comprising:
a push pulse transmission unit that transmits the push pulse;
a displacement detection unit that transmits detection waves to the subject a plurality of times after the push pulse is transmitted, receives reflected detection waves corresponding to the detection waves from the subject, generates a plurality of received signals in time series, and detects displacement of the tissues in the subject due to the shear waves caused by the push pulse at each time when the reflected detection waves are received;
an elasticity measurement unit that analyzes the propagation state of the shear waves in the region of interest on the basis of a detection result of the displacement detection unit and measures elasticity of each tissue in the subject;
a probe movement detection unit that detects a moving speed of the ultrasound probe;
a sequence holding unit that holds a plurality of operation sequences defining a series of operations performed by the push pulse transmission unit, the displacement detection unit, and the elasticity measurement unit in cooperation with each other which enables the elasticity measurement unit to measure the elasticity; and
a sequence selection unit that selects one operation sequence from the plurality of operation sequences held by the sequence holding unit on the basis of a detection result of the probe movement detection unit.

2. The ultrasound diagnostic apparatus according to claim 1,
wherein, when the moving speed of the ultrasound probe is greater than a predetermined speed, the sequence selection unit selects an operation sequence such that the influence of the moving speed of the ultrasound probe on the measurement result of the elasticity measurement unit is small.

3. The ultrasound diagnostic apparatus according to claim 2, further comprising:
an elastic image generation unit that generates one elastic image indicating the elasticity of each tissue in the subject for one operation sequence, on the basis of the measurement result of the elasticity measurement unit,
wherein, when the moving speed of the ultrasound probe is greater than the predetermined speed, the sequence selection unit selects an operation sequence such that a frame rate of the elastic image is improved.

4. The ultrasound diagnostic apparatus according to claim 2,
wherein the operation sequence includes one push pulse transmission operation, an operation of detecting displacement corresponding to the push pulse, and an operation of measuring elasticity based on the detected displacement, and
when the moving speed of the ultrasound probe is greater than the predetermined speed, the sequence selection unit selects an operation sequence such that at least one of a spatial throughput and a temporal throughput is reduced in the detection of the displacement by the displacement detection unit and the time required for one operation sequence is reduced.

5. The ultrasound diagnostic apparatus according to claim 2,
wherein the operation sequence includes two or more sub-sequences,
each sub-sequence includes one push pulse transmission operation, an operation of detecting displacement corresponding to the push pulse, and an operation of measuring elasticity based on the detected displacement,
a position on which the push pulse is focused varies depending on the sub-sequence,
the operation sequence further includes a process of measuring the elasticity of each tissue in the subject on the basis of the elasticity measured for each sub-sequence, and
the sequence selection unit selects an operation sequence such that at least one of (1) when the moving speed of the ultrasound probe is greater than the predetermined speed, at least one of a spatial throughput and a temporal throughput is reduced in the detection of the displacement by the displacement detection unit and the time required for one sub-sequence is reduced and (2) the number of sub-sequences included in one operation sequence is reduced is satisfied.

6. The ultrasound diagnostic apparatus according to claim 2,
wherein the operation sequence is defined by parameters for designating a temporal resolution indicating how frequently the displacement detection unit generates a received signal and detects displacement and a spatial resolution when the displacement detection unit detects the displacement, and
when the moving speed of the ultrasound probe is greater than the predetermined speed, the sequence selection unit selects an operation sequence such that at least one of the temporal resolution and the spatial resolution is reduced.

7. The ultrasound diagnostic apparatus according to claim 2,
wherein, when the moving speed of the ultrasound probe is equal to or less than the predetermined speed, the sequence selection unit selects an operation sequence such that a spatial resolution of the received signal generated by the displacement detection unit is improved.

8. The ultrasound diagnostic apparatus according to claim 2,
wherein, in a case in which the sequence selection unit is set so as to select a first operation sequence when the moving speed of the ultrasound probe is greater than a first speed and to select a second operation sequence when the moving speed of the ultrasound probe is equal to or less than the first speed, when the moving speed of the ultrasound probe is greater than a second speed greater than the first speed, the sequence selection unit selects a third operation sequence, instead of the first operation sequence.

9. The ultrasound diagnostic apparatus according to claim 1,
wherein, when the moving speed of the ultrasound probe is greater than a first speed, the sequence selection unit selects an operation sequence which does not start until the moving speed of the ultrasound probe is equal to or less than a second speed.

10. The ultrasound diagnostic apparatus according to claim 9,
wherein the second speed is equal to or less than the first speed.

11. The ultrasound diagnostic apparatus according to claim 1,
wherein the sequence selection unit selects an operation sequence such that the displacement detection unit generates a reference signal before the push pulse is transmitted and detects displacement using a difference between the reference signal and the received signal when the moving speed of the ultrasound probe is equal to or less than a predetermined speed, and the displacement detection unit detects displacement, using a difference between a plurality of received signals which are arranged in time series as an amount of change in displacement, when the moving speed of the ultrasound probe is greater than the predetermined speed.

12. The ultrasound diagnostic apparatus according to claim 1,
wherein the probe movement detection unit detects the moving speed of the ultrasound probe on the basis of the received signal acquired by the ultrasound probe.

13. The ultrasound diagnostic apparatus according to claim 1,
wherein the probe movement detection unit detects the moving speed of the ultrasound probe on the basis of a signal acquired from a sensor that is provided inside or outside the ultrasound probe.

14. An ultrasound signal processing method that transmits a push pulse for focusing ultrasonic waves on a specific part of a subject to physically press tissues in the specific part using an ultrasound probe, repeatedly transmits and receives the ultrasonic waves to and from the subject using the ultrasound probe, and detects a propagation state of shear waves generated from the pressed tissues of the specific part, which are a vibration source, in a region of interest set in the subject, comprising:
detecting a moving speed of the ultrasound probe;
selecting an operation sequence defining a series of operations for measuring elasticity in the region of interest in the subject from a plurality of operation sequences which are held in advance, on the basis of the moving speed of the ultrasound probe; and
transmitting the push pulse, transmitting detection waves to the subject a plurality of times after the push pulse is transmitted, receiving reflected detection waves corresponding to the detection waves from the subject, generating a plurality of received signals in time series, detecting displacement of the tissues in the subject due to the shear waves caused by the push pulse at each time when the reflected detection waves are received, and analyzing the propagation state of the shear waves in the region of interest on the basis of the displacement of the tissues in the subject to measure elasticity of each tissue in the subject, according to the selected operation sequence.
